SEQUENCE LISTING (8 sequences)

SEQ ID NO: 1 - protein, 162 aa, Artificial Sequence
  Human IL-21 Canonical Amino Acid Sequence
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 162 aa, Artificial Sequence
  Human IL-15 Canonical Amino Acid Sequence
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 254 aa, Artificial Sequence
  Human 4-1BBL Canonical Amino Acid Sequence
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 183 aa, Artificial Sequence
  Human OX40L Canonical Amino Acid Sequence
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - DNA, 486 bp, Artificial Sequence
  Human IL-21 Canonical Nucleic Acid Sequence
atgcgcagcagcccgggcaacatggaacgcattgtgatttgcctgatggtgatttttctgggcaccctggtgcataaaagcagcagccagggccaggatcgccatatgattcgcatgcgccagctgattgatattgtggatcagctgaaaaactatgtgaacgatctggtgccggaatttctgccggcgccggaagatgtggaaaccaactgcgaatggagcgcgtttagctgctttcagaaagcgcagctgaaaagcgcgaacaccggcaacaacgaacgcattattaacgtgagcattaaaaaactgaaacgcaaaccgccgagcaccaacgcgggccgccgccagaaacatcgcctgacctgcccgagctgcgatagctatgaaaaaaaaccgccgaaagaatttctggaacgctttaaaagcctgctgcagaaaatgattcatcagcatctgagcagccgcacccatggcagcgaagatagc

SEQ ID NO: 6 - DNA, 486 bp, Artificial Sequence
  Human IL-15 Canonical Nucleic Acid Sequence
atgcgcattagcaaaccgcatctgcgcagcattagcattcagtgctatctgtgcctgctgctgaacagccattttctgaccgaagcgggcattcatgtgtttattctgggctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 7 - DNA, 762 bp, Artificial Sequence
  Human 4-1BBL Canonical Nucleic Acid Sequence
atggaatatgcgagcgatgcgagcctggatccggaagcgccgtggccgccggcgccgcgcgcgcgcgcgtgccgcgtgctgccgtgggcgctggtggcgggcctgctgctgctgctgctgctggcggcggcgtgcgcggtgtttctggcgtgcccgtgggcggtgagcggcgcgcgcgcgagcccgggcagcgcggcgagcccgcgcctgcgcgaaggcccggaactgagcccggatgatccggcgggcctgctggatctgcgccagggcatgtttgcgcagctggtggcgcagaacgtgctgctgattgatggcccgctgagctggtatagcgatccgggcctggcgggcgtgagcctgaccggcggcctgagctataaagaagataccaaagaactggtggtggcgaaagcgggcgtgtattatgtgttttttcagctggaactgcgccgcgtggtggcgggcgaaggcagcggcagcgtgagcctggcgctgcatctgcagccgctgcgcagcgcggcgggcgcggcggcgctggcgctgaccgtggatctgccgccggcgagcagcgaagcgcgcaacagcgcgtttggctttcagggccgcctgctgcatctgagcgcgggccagcgcctgggcgtgcatctgcataccgaagcgcgcgcgcgccatgcgtggcagctgacccagggcgcgaccgtgctgggcctgtttcgcgtgaccccggaaattccggcgggcctgccgagcccgcgcagcgaa

SEQ ID NO: 8 - DNA, 549 bp, Artificial Sequence
  Human OX40L Canonical Nucleic Acid Sequence
atggaacgcgtgcagccgctggaagaaaacgtgggcaacgcggcgcgcccgcgctttgaacgcaacaaactgctgctggtggcgagcgtgattcagggcctgggcctgctgctgtgctttacctatatttgcctgcattttagcgcgctgcaggtgagccatcgctatccgcgcattcagagcattaaagtgcagtttaccgaatataaaaaagaaaaaggctttattctgaccagccagaaagaagatgaaattatgaaagtgcagaacaacagcgtgattattaactgcgatggcttttatctgattagcctgaaaggctattttagccaggaagtgaacattagcctgcattatcagaaagatgaagaaccgctgtttcagctgaaaaaagtgcgcagcgtgaacagcctgatggtggcgagcctgacctataaagataaagtgtatctgaacgtgaccaccgataacaccagcctggatgattttcatgtgaacggcggcgaactgattctgattcatcagaacccgggcgaattttgcgtgctg

(19) United States
(12) Patent Application Publication
(10) Pub. No.: US 2024/0216426 A1
(43) Pub. Date: Jul. 4, 2024

(54) RECOMBINANT ANTIGEN PRESENTING CELLS

(71) Applicant: TAKEDA PHARMACEUTICAL COMPANY LIMITED, Osaka (JP)

(72) Inventors: Nandhu SOBHANA, Cambridge, MA (US); Alisha BOTHUN, Cambridge, MA (US); Gil BENEZER, Cambridge, MA (US); Shruti BADKAR, Cambridge, MA (US); Rabi MISHRA, Cambridge, MA (US); Hui-Hsin CHANG, Cambridge, MA (US); Wanwen LI, Cambridge, MA (US); Mansi SONI, Cambridge, MA (US); Lan CAO, Cambridge, MA (US); Xi SHl, Cambridge, MA (US); Jane YEH, Cambridge, MA (US); Mengyao LUO, Cambridge, MA (US); Manoj GUPTA, Cambridge, MA (US); Akito NAKAMURA, Cambridge, MA (US); Doanh MAI, Cambridge, MA (US); Mei Rosa NG, Cambridge, MA (US)

(73) Assignee: TAKEDA PHARMACEUTICAL COMPANY LIMITED, Osaka (JP)

(21) Appl. No.: 18/557,559

(22) PCT Filed: Apr. 27, 2022
(86) PCT No.: PCT/IB2022/053925

§ 371 Date: Oct. 26, 2023

Related U.S. Application Data
(60) Provisional application No. 63/180,530, filed on Apr. 27, 2021.

(51) Int. Cl. A61K 35/15; A61K 39/00; C07K 14/54; C07K 14/705

(52) U.S. Cl. CPC A61K 35/15; A61K 39/463; C07K 14/54; C07K 14/5443; C07K 14/70575; C07K 2319/03; C07K 2319/30; C12N 2501/2315; C12N 2501/2321; C12N 2506/45; C12N 2510/00

(57) ABSTRACT

The present disclosure provides recombinant antigen presenting cells and methods of use thereof in the culture and expansion of immune cells ex vivo. In some aspects, immune cells expanded through co-culture of the recombinant antigen presenting cells disclosed herein are administered to a subject to treat a disease or condition in the subject, e.g., to treat a cancer.

### CROSS-REFERENCE TO RELATED APPLICATIONS

This application claims priority benefit of U.S. Provisional Application No. 63/180,530 filed Apr. 27, 2021, which is incorporated by reference herein in its entirety.

### REFERENCE TO SEQUENCE LISTING SUBMITTED ELECTRONICALLY VIA EFS-WEB

The content of the electronically submitted sequence listing (Name: 3817_085 PC01_Seqlisting_ST25.txt, Size: 11,031 bytes; and Date of Creation: Apr. 26, 2022) submitted in this application is incorporated herein by reference in its entirety.

### FIELD

The present disclosure relates to antigen presenting cells and the use thereof in the culture and expansion of immune cells ex vivo.

### BACKGROUND

Cell based immunotherapy is a rapidly developing field of research for the development of novel and improved methods of treating various diseases, including cancer. Isolating and expanding immune cells for an immunotherapy requires supplementing the cells with various factors to improve their survival, expansion, and ultimate efficacy. Antigen presenting cells are often used as a vehicle for delivering these factors to immune cells cultured ex vivo. However, populations of immune cells isolated from a subject consist of a variety of cell types, including alpha/beta T cells, gamma/delta T cells, NK cells, and B cells. Certain types of immune cells, for example, gamma/delta T cells and NK cells, represent a small fraction of the total population of immune cells, making it challenging to arrive at cell numbers that are suitable for a cell-based immunotherapy.

As such, there remains a need in the art for methods of expanding populations of immune cells, and in particular, cells that represent a small fraction of the total cell population, e.g., NK cells and gamma/delta T cells in particular.

### BRIEF SUMMARY

The present disclosure provides, in part, recombinant antigen presenting cells that are useful for isolation and/or expansion of immune cells suitable for cell-based immunotherapy and in particular, immune cells which represent a small fraction of the total cell population. Some aspects of the present disclosure are directed to a recombinant antigen presenting cell (RAPC), which comprises (i) one or more nucleic acid molecules encoding IL-21 and (ii) one or more nucleic acid molecules encoding OX40L. In some aspects, the RAPC further comprises (iii) one or more nucleic acid molecule encoding 4-1BBL. In some aspects, the RAPC further comprises (iv) one or more nucleic acid molecules encoding IL-15. In some aspects, the RAPC expresses (i) IL-21 and (ii) OX40L. In some aspects, the RAPC expresses (iii) 4-1BBL. In some aspects, the RAPC expresses (iv) IL-15.

In some aspects, the IL-21 is a fusion protein comprising a human IL-21 polypeptide and an Fc region of an immunoglobulin. In some aspects, the IL-21 comprises (i) the human IL-21 polypeptide, (ii) a hinge region, (iii) the Fc region of an immunoglobulin, and (iv) a transmembrane domain. In some aspects, the IL-15 is a fusion protein comprising a human IL-15 polypeptide and an Fc region of an immunoglobulin. In some aspects, the IL-15 comprises (i) the human IL-15 polypeptide, (ii) a hinge region, (iii) the Fc region of an immunoglobulin, and (iv) a transmembrane domain. In some aspects, the Fc region is an IgG1, IgG2, IgG3, or IgG4 Fc region. In some aspects, the Fc region is an IgG4 Fc region. In some aspects, the hinge region comprises an immunoglobulin hinge region or a modified immunoglobulin hinge region. In some aspects, the hinge region comprises an IgG1, IgG2, IgG3, IgG4 or CD8 hinge region. In some aspects, the transmembrane domain comprises a CD4 or CD8 transmembrane domain.

In some aspects, the RAPC comprises a dendritic cell, an engineered clonal cell line, a naturally derived cancer cell line, a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support or any combination thereof. In some aspects, the RAPC is a K562 cell. In some aspects, the RAPC is a genetically modified K562 cell.

Some aspects of the present disclosure are directed to a population of RAPCs comprising an RAPC disclosed herein.

Some aspects of the present disclosure are directed to a population of RAPCs comprising a first RAPC and a second RAPC, wherein: (a) the first RAPC and the second RAPC comprise one or more nucleic acid molecules encoding IL-21 and one or more nucleic acid molecules encoding OX40L; or (b) the first RAPC comprises one or more nucleic acid molecules encoding IL-21, and wherein the second RAPC expresses one or more nucleic acid molecules encoding OX40L. In some aspects, the first RAPC, the second RAPC, or both further comprises one or more nucleic acid molecules encoding 4-1BBL. In some aspects, the first RAPC, the second RAPC, or both further comprises one or more nucleic acid molecules encoding IL-15. In some aspects, a population of RAPCs further comprises a third RAPC. In some aspects, the third RAPC comprises one or more nucleic acid molecules encoding 4-1BBL. In some aspects, the third RAPC comprises one or more nucleic acid molecules encoding IL-15. In some aspects, a population of RAPCs further comprises a fourth RAPC. In some aspects, the fourth RAPC comprises one or more nucleic acid molecules encoding IL-15.

In some aspects, the first RAPC, the second RAPC, the third RAPC, the fourth RAPC, or any combination thereof comprises a dendritic cell, an engineered clonal cell line, a naturally derived cancer cell line, a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support or any combination thereof. In some aspects, the first RAPC, the second RAPC, the third RAPC, the fourth RAPC, or any combination thereof is a K562 cell. In some aspects, the first RAPC, the second RAPC, the third RAPC, the fourth RAPC, or any combination thereof is a genetically modified K562 cell.

Some aspects of the present disclosure are directed to a method of expanding a population of immune cells ex vivo, comprising contacting the population of immune cells with an RAPC disclosed herein or a population of RAPCs disclosed herein.

Some aspects of the present disclosure are directed to a method of expanding a population of immune cells ex vivo, comprising contacting the population of immune cells with an RAPC, wherein the RAPC expresses IL-21, OX40L, 4-1BBL, and IL-15.

In some aspects, the immune cells comprise an αβ T cell, a B cell, a natural killer (NK) cell, a tumor infiltrating lymphocyte (TIL), a pan γδ T cell, a Vδ1 γδ T cell, a Vδ2 γδ T cell, a Vδ3 γδ T cell or any combination thereof. In some aspects, the T cell is selected from the group consisting of a naïve T cell, a stem cell memory T cell (Tscm), a central memory T cell (Tcm), an effector T cell, an effector memory T cell (Tem), a cytotoxic T cell, a helper T cell, and any combination thereof.

In some aspects, the population of immune cells comprises one or more genetically modified immune cells. In some aspects, the genetically modified immune cells comprise a chimeric antigen receptor (CAR), an engineered T cell receptor (TCR), or a combination thereof. In some aspects, the CAR or the TCR is capable of binding one or more antigens present on a tumor cell. In some aspects, the CAR or the TCR is capable of binding an antigen selected from the group consisting of CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), and any combination thereof.

In some aspects, the population of immune cells is expanded until the total number of immune cells in the population of immune cells is at least about 1×10⁷ to at least about 1×10¹², at least about 1×10⁸ to at least about 1×10¹², at least about 1×10⁹ to at least about 1×10¹², at least about 1×10⁸ to at least about 1×10¹¹, at least about 1×10⁹ to at least about 1×10¹¹, at least about 1×10¹⁰ to at least about 1×10¹¹, at least about 1×10⁷ to at least about 1×10¹⁰, at least about 1×10⁸ to at least about 1×10¹⁰, or at least about 1×10⁹ to at least about 1×10¹⁰ immune cells. In some aspects, the population of immune cells is expanded until the total number of immune cells in the population of immune cells is at least about 1×10⁷, at least about 5×10⁷, at least about 1×10⁸, at least about 5×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, at least about 5×10¹¹, at least about 6×10¹¹, at least about 7×10¹¹, at least about 8×10¹¹, at least about 9×10¹¹, or at least about 1×10¹² immune cells.

In some aspects, the population of immune cells are obtained from a donor subject. In some aspects, the donor subject is a human. In some aspects, the donor subject is afflicted with a cancer.

In some aspects, the method further comprises purifying an expanded population of immune cells, for example, by using positive or negative selection or any other suitable means for obtaining a substantially pure population of expanded immune cells, which can subsequently be used for cell therapy. In some aspects, the expanded population of immune cells comprises a population of alpha/beta T cells, gamma/delta T cells, NK cells or a combination thereof.

In some aspects, the method further comprises administering the expanded population of immune cells to a subject in need thereof.

Some aspects of the present disclosure are directed to a method of treating a disease or condition in a subject in need thereof, comprising (i) expanding a population of immune cells ex vivo by contacting the population of immune cells with an RAPC disclosed herein or a population of RAPCs disclosed herein; (ii) purifying an expanded population of immune cells; and (iii) administering the purified population of immune cells to the subject.

In some aspects, the subject is afflicted with a cancer. In some aspects, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. In some aspects, the cancer comprises a lymphoma or a leukemia.

Some aspects of the present disclosure are directed to a method of expanding a population of immune cells, e.g., T cells and/or NK cells, ex vivo, comprising contacting the population of immune cells with interleukin-21 (IL-21) and OX40L. In some aspects, the method further comprises contacting the population of immune cells with an antigen presenting cell (APC) disclosed herein. Some aspects of the present disclosure are directed to a method of expanding a population of immune cells, e.g., T cells and/or NK cells, ex vivo, comprising contacting the population of immune cells with an APC, IL-21, and OX40L. In some aspects, the method further comprises contacting the population of immune cells with 4-1BB ligand (4-1BBL). In some aspects, the method further comprises contacting the population of immune cells with interleukin-15 (IL-15).

In some aspects, the APC expresses IL-21, OX40L, 4-1BBL, IL-15, or any combination thereof. In some aspects, the APC expresses IL-21. In some aspects, the APC expresses OX40L. In some aspects, the APC expresses 4-1BBL. In some aspects, the APC expresses IL-15. In some aspects, the APC expresses IL-21 and OX40L, wherein the population of immune cells are cultured in a medium, and wherein the medium comprises 4-1BBL and IL-15. In some aspects, the APC expresses IL-21, OX40L, and 4-1BBL, wherein the population of immune cells are cultured in a medium, and wherein the medium comprises IL-15. In some aspects, the APC expresses IL-21, OX40L, and IL-15, wherein the population of immune cells are cultured in a medium, and wherein the medium comprises 4-1BBL.

In some aspects, the APC comprises a recombinant APC (RAPC). In some aspects, the RAPC comprises a dendritic cell, an engineered clonal cell line, a naturally derived cancer cell line, a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support or any combination thereof.

Some aspects of the present disclosure are directed to a method of expanding a population of immune cells, e.g., T cells and/or NK cells, ex vivo, comprising contacting the population of immune cells with IL-21 and a first APC, wherein the first APC expresses OX40L. In some aspects, the population of immune cells is cultured in a medium, wherein the medium comprises the IL-21. In some aspects, the IL-21 is associated with a solid support, e.g., a bead. In some aspects, the IL-21 is expressed by a second APC. In some aspects, the first RAPC and the second APC are different. In some aspects, the first APC and the second APC are the same.

In some aspects, the method further comprises contacting the population of immune cells with OX40L. In some aspects, the population of immune cells is cultured in a medium, wherein the medium comprises the OX40L. In some aspects, the OX40L is associated with a solid support, e.g., a bead. In some aspects, the OX40L is expressed by a third APC. In some aspects, the third APC is different from the first APC and the second APC. In some aspects, the third APC is the same as the first APC, wherein the third APC is the same as the second APC, or wherein the third APC is the same as the first APC and the second APC.

In some aspects, the method further comprises contacting the population of immune cells with IL-15. In some aspects, the population of immune cells is cultured in a medium, wherein the medium comprises the IL-15. In some aspects, the IL-15 is associated with a solid support, e.g., a bead. In some aspects, the IL-15 is expressed by a fourth APC. In some aspects, the fourth APC is different from the first APC, the second APC, and the third APC. In some aspects, the fourth APC is the same as the first APC; wherein the fourth APC is the same as the second APC; wherein the fourth APC is the same as the third APC; wherein the fourth APC is the same as the first APC and the second APC; wherein the fourth APC is the same as the second APC and the third APC; wherein the fourth APC is the same as the first APC and the third APC; or wherein the fourth APC is the same as the first APC, the second APC, and the third APC. In some aspects, one or more of the first APC, the second APC, the third APC, or the fourth APC comprises a dendritic cell, an engineered clonal cell line, a naturally derived cancer cell line, a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support or any combination thereof.

Some aspects of the present disclosure are directed to a method of expanding a population of immune cells ex vivo, comprising contacting the population of immune cells with an APC, wherein the APC expresses IL-21, OX40L, 4-1BBL, and IL-15. In some aspects, the APC is an RAPC. In some aspects, the RAPC comprises a dendritic cell, an engineered clonal cell line, a naturally derived cancer cell line, a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support or any combination thereof.

In some aspects, the IL-21 is a fusion protein comprising a human IL-21 polypeptide and an Fc region of an immunoglobulin. In some aspects, the IL-21 comprises (i) the human IL-21 polypeptide, (ii) a hinge region, (iii) the Fc region of an immunoglobulin, and (iv) a transmembrane domain. In some aspects, the IL-15 is a fusion protein comprising a human IL-15 polypeptide and an Fc region of an immunoglobulin. In some aspects, the IL-15 comprises (i) the human IL-15 polypeptide, (ii) a hinge region, (iii) the Fc region of an immunoglobulin, and (iv) a transmembrane domain. In some aspects, the Fc region is an IgG1, IgG2, IgG3, or IgG4 Fc region. In some aspects, the Fc region is an IgG4 Fc region. In some aspects, the hinge region comprises an immunoglobulin hinge region or a modified immunoglobulin hinge region. In some aspects, the hinge region comprises an IgG1, IgG2, IgG3, IgG4 or CD8 hinge region. In some aspects, the transmembrane domain comprises a CD4 or CD8 transmembrane domain.

In some aspects, the population of immune cells is expanded until the total number of immune cells in the population of immune cells is at least about 1×10⁷ to at least about 1×10¹², at least about 1×10⁸ to at least about 1×10¹², at least about 1×10⁹ to at least about 1×10¹², at least about 1×10⁸ to at least about 1×10¹¹, at least about 1×10⁹ to at least about 1×10¹¹, at least about 1×10¹⁰ to at least about 1×10¹¹, at least about 1×10⁷ to at least about 1×10¹⁰, at least about 1×10⁸ to at least about 1×10¹⁰, or at least about 1×10⁹ to at least about 1×10¹⁰ immune cells. In some aspects, the population of immune cells is expanded until the total number of immune cells in the population of immune cells is at least about 1×10⁷, at least about 5×10⁷, at least about 1×10⁸, at least about 5×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, at least about 5×10¹¹, at least about 6×10¹¹, at least about 7×10¹¹, at least about 8×10¹¹, at least about 9×10¹¹, or at least about 1×10¹² immune cells.

In some aspects, the population of immune cells are obtained from a donor subject. In some aspects, the donor subject is a human. In some aspects, the donor subject is afflicted with a cancer. In some aspects, the method further comprises administering the population of immune cells to a subject in need thereof.

### BRIEF DESCRIPTION OF THE DRAWINGS/FIGURES

FIGS. 1A-1D are schematic illustrations of the construct designs for the generation of lentivirus comprising transgenes encoding 4-1BBL (FIG. 1A), OX40L (FIG. 1B), IL-15-CD8 fusion protein (FIG. 1C), and IL-21-CD8 fusion protein (FIG. 1D). FIG. 1E is a flowchart description of the process for generating recombinant APCs.

FIGS. 2A-2B are bar graphs showing the effect of RAPC2 and RAPC4 co-incubation on cell expansion, phenotype, and cytotoxicity against Nalm6 and Raji tumor cells, of cord blood (CB) and peripheral blood (PB)-derived NK cells. NK cells were negatively selected via immunomagnetic bead separation from a cord blood or leukapheresis collection and were coincubated with mitomycin C-pretreated RAPC2 or RAPC4 cells in RPMI (Gibco) containing 10% FBS (Corning) in the presence of IL-2 (Prometheus). Cell cultures were refreshed with half-volume medium changes supplemented with IL-2 every two days. Cells were cultured for 4 weeks in G-Rex 6M well plate (Wilson Wolf). Cell growth and viability were monitored by NC-200 (ChemoMetec) during every activation round. FIG. 2A provides a cell surface phenotype (CD3-CD56+) comparison of NK cells at day 28 of culture analyzed by flow cytometry. FIG. 2B shows the sum percentages of dead target cells at multiple E:T ratios (starting from 5:1 to 0.15:1) for CB-NK or PB-NK cells co-incubated with Nalm6 or Raji tumor cells for 24 hours.

FIG. 3 shows cytotoxicity of cord blood (CB) and peripheral blood (PB)-derived gamma/delta T (gdT) cells by co-incubation with RAPC2 and RAPC4 feeder cells. gdT cells were positively selected via immunomagnetic bead separation from a cord blood or leukapheresis collection. Cells were co-incubated with mitomycin C-pretreated RAPC2 or RAPC4 feeder cells in RPMI (Gibco) containing 10% FBS (Corning) in the presence of IL-2 (Peprotech) and IL-21 (Peprotech). Cell cultures were refreshed with half-volume medium changes supplemented with IL-2 and IL-21 every two days. Cells were cultured for 4 weeks in G-Rex 6M well plate (Wilson Wolf). Cell growth and viability were monitored by NC-200 (ChemoMetec) during every 7 day of activation round. CB-gdT expanded by RAPC4 showed the intrinsic cytolytic capacity against multiple tumor cell lines. CB-gdT cells were co-incubated with tumor cells at different E:T ratios (starting from 10:1 to 0.3:1) for 24 hours.

FIG. 4 is a graphical representation of the effect of RAPC1 and RAPC4 feeder cell lines on cell expansion of human NK cells. RAPC1 and RAPC4 cells were X-Ray irradiated (100 Gy) immediately prior to co-incubation with NK cells in IMDM (Gibco) containing 15% FBS (Gibco). RAPC1 co-cultures were supplemented with IL-2, IL-7, and IL-15; RAPC4 co-cultures were supplemented with IL-2 and IL-7. Feeder cells were added at a ratio of 2:1 (feeders:NK) for three days of activation followed by expansion for four days in G-Rex 24 well plates with a 60% medium change on day 5 supplemented with cytokines. Activation was repeated every 7 days. Cell growth and viability were monitored by NC-200 (ChemoMetec) during every 7 day of activation round.

FIG. 5 is a bar graph illustrating NK cell surface marker expression following co-culture with RAPC4 for 7 and 11 days. RAPC4 cells were X-Ray irradiated (100 Gy) immediately prior to co-incubation with NK cells in IMDM (Gibco) containing 15% FBS (Gibco) supplemented with IL-2 and IL-7. Feeder cells were added at a ratio of 2:1 (feeders:NK) for three days activation followed by transfer of cultures to G-Rex 24 well plates for expansion for four days, with a 60% medium change on day 5 supplemented with cytokines. NK cells were harvested after 7 and 11 days of culture and analyzed by flow cytometry for surface expression of CD3, CD56, CD30, CD16, CD94, NKG2D, and NKp46. Values represent the percentage of all live cells that stained positively for the respective marker.

FIGS. 6A-6E are line graphs illustrating RAPC4-activated NK cell expansion and tumor cell killing. RAPC4 cells were X-Ray irradiated (100 Gy) immediately prior to co-incubation with NK cells at a ratio of 2:1. NK cells activated with irradiated RAPC4 were subsequently transduced with 7 different CAR lentiviral vectors or left untransduced (UTD) to examine expansion post-transduction. On the 6th day of the second round of activation, NK cells were harvested and incubated with four tumor cell lines (Capan2 (FIG. 6A), GSU (FIG. 6B), HCT116 (FIG. 6C), and RAJI (FIG. 6E) (ATCC) and one normal endothelial cell line control (HUVEC; FIG. 6D) at an E:T ratio of 3:1, 1:1, 0.3:1, and 0.1:1. Percent cytolysis was evaluated 24 hours post co-culture using the Cell Titer Glo Assay (Promega).

### DETAILED DESCRIPTION

Some aspects of the present disclosure are directed to recombinant antigen-presenting cells (RAPCs). In some aspects, the RAPC comprises (i) one or more one or more nucleic acid molecules encoding IL-21 and (ii) one or more nucleic acid molecules encoding OX40L. In some aspects, the RAPC comprises (i) one or more one or more nucleic acid molecules encoding IL-21, (ii) one or more nucleic acid molecules encoding OX40L, and (iii) one or more nucleic acid molecule encoding 4-1BBL. In some aspects, the RAPC comprises (i) one or more one or more nucleic acid molecules encoding IL-21, (ii) one or more nucleic acid molecules encoding OX40L, and (iii) one or more nucleic acid molecules encoding IL-15. In some aspects, the RAPC comprises (i) one or more one or more nucleic acid molecules encoding IL-21, (ii) one or more nucleic acid molecules encoding OX40L, (iii) one or more nucleic acid molecule encoding 4-1BBL, and (iv) one or more nucleic acid molecules encoding IL-15.

Other aspects of the present disclosure are directed to methods of expanding a population of immune cells ex vivo, comprising contacting the population of immune cells with an RAPC or a population thereof disclosed herein. In some aspects, the immune cells comprise an αβ T cell, a B cell, a natural killer (NK) cell, a tumor infiltrating lymphocyte (TIL), a pan γδ T cell, a Vδ1 γδ T cell, a Vδ2 γδ T cell, a Vδ3 γδ T cell, or any combination thereof. In some aspects, the population of immune cells comprises one or more genetically modified immune cells. In some aspects, the genetically modified immune cells comprise a chimeric antigen receptor (CAR), an engineered T cell receptor (TCR), or a combination thereof.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

As described herein, any concentration range, percentage range, ratio range or integer range is to be understood to include the value of any integer within the recited range and, when appropriate, fractions thereof (such as one tenth and one hundredth of an integer), unless otherwise indicated.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). In addition, “or” is used mean an open list of the components in the list. For example, “wherein X comprises A or B” means X comprises A, X comprises B, X comprises A and B, or X comprises A or B and any other components.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “activated immune cells,” “activated T cells,” and “activated NK cells” refer to, among other things, immune cells, e.g., T cells and/or NK cells, that are undergoing cell division.

An “antigen” refers to any molecule, e.g., a peptide, that provokes an immune response or is capable of being bound by a TCR. The immune response may involve antibody production, the activation of specific immunologically-competent cells, or a combination thereof. A person of skill in the art would readily understand that any macromolecule, including virtually all proteins or peptides, can serve as an antigen. An antigen can be endogenously expressed, i.e. expressed by genomic DNA, or can be recombinantly expressed. An antigen and/or an epitope can be specific to a certain tissue, such as a cancer cell, or it can be broadly expressed. In addition, fragments of larger molecules can act as antigens. In one aspect, antigens are tumor antigens.

An “antigen-presenting cell” or “APC,” as used herein, refers to a cell or a cell-like antigen-presenting surface that expresses one or more antigen. In some aspects, the antigen is displayed on the surface of the APC. Non-limiting examples of APCs include a dendritic cell, an engineered clonal cell line, a naturally derived cancer cell line, or any combination thereof. Non-limiting examples of a cell-like antigen-presenting surface include a bead-based artificial antigen presenting cell, an artificial lipid vesicle or other lipid bilayer containing system, an exosome, a solid support including but not limited to, for example, a bead, a spherical or non-spherical nanoparticle support, a carbon nanotube support, a magnetic particle support, a filamentous polymer support, a two dimensional support, or any combination thereof. In some aspects, an antigen-presenting cell described herein is a K562 cell. In some aspects, the antigen-presenting cell described herein is a K562 cell, which is modified to express one or more of IL-21, OX40L, 4-1BBL, and IL-15. In some aspects, antigen-presenting cell described herein is a K562 cell, which is modified to express IL-21, OX40L, 4-1BBL, and IL-15. In some aspects, an antigen-presenting cell described herein is a 721.221 cell. See, e.g., Yang et al., Mol. Ther.: Methods & Clinical Development 18:428 (2020). In some aspects, the antigen-presenting cell described herein is a 721.221 cell, which is modified to express one or more of IL-21, OX40L, 4-1BBL, and IL-15. In some aspects, antigen-presenting cell described herein is a 721.221 cell, which is modified to express IL-21, OX40L, 4-1BBL, and IL-15. In some aspects, an antigen-presenting cell described herein is a Hut78 cell. See, e.g., Min et al., Cellular & Molecular Immunology 19:296-98 (2022). In some aspects, the antigen-presenting cell described herein is a Hut78 cell, which is modified to express one or more of IL-21, OX40L, 4-1BBL, and IL-15. In some aspects, antigen-presenting cell described herein is a Hut78 cell, which is modified to express IL-21, OX40L, 4-1BBL, and IL-15.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “autologous” refers to any material, e.g., an immune cell, derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some embodiments, the methods of the present invention can be used to reduce the tumor size of a tumor derived from, for example, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, breast cancer, prostate cancer, lung cancer (e.g., non-small cell lung cancer (NSCLC) or small cell lung cancer (SCLC)), Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. The particular cancer can be responsive to chemo- or radiation therapy or the cancer can be refractory. A refractory cancer refers to a cancer that is not amendable to surgical intervention, and the cancer is either initially unresponsive to chemo- or radiation therapy or the cancer becomes unresponsive over time.

It is understood that wherever aspects are described herein with the language “comprising,” otherwise analogous aspects described in terms of “consisting of” and/or “consisting essentially of” are also provided.

“Co-stimulatory ligand,” as the term is used herein, includes a molecule on an antigen presenting cell (e.g., an aAPC, dendritic cell, B cell, and the like) that specifically binds a cognate co-stimulatory molecule on an immune cell, e.g., a T cell or an NK cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible COStimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell, e.g., a T cell and/or an NK cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83. In some embodiment, the co-stimulatory molecules include one or more of IL-21 (e.g., membrane bound IL-21), 4-1BBL, OX40L and IL-15 (e.g, membrane bound IL-15), or any combinations thereof. In a particular embodiment, a RAPC described herein includes each of membrane bound IL-21, membrane bound IL-15, OX40L and 4-1BBL.

A “co-stimulatory molecule” refers to the cognate binding partner on an immune cell, e.g., a T cell or an NK cell, that specifically binds with a co-stimulatory ligand, thereby mediating a co-stimulatory response by the immune cell, such as, but not limited to, proliferation. Co-stimulatory molecules include, but are not limited to an MHC class I molecule, BTLA and a Toll ligand receptor.

A “cytokine,” as used herein, refers to a non-antibody protein that is released by one cell in response to contact with a specific antigen, wherein the cytokine interacts with a second cell to mediate a response in the second cell. A cytokine can be endogenously expressed by a cell, added to a cell in culture, administered to a subject, or any combination thereof. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, IL-21, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (SICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1a (MIP-1a, MIP-1a), MIP-Ib (MIP-1b), gamma-induced protein 10 (IP-10), and thymus and activation regulated chemokine (TARC or CCL17).

Other examples of cytokines include, but are not limited to chemokine (C—C motif) ligand (CCL) 1, CCL5, monocyte-specific chemokine 3 (MCP3 or CCL7), monocyte chemoattractant protein 2 (MCP-2 or CCL8), CCL13, IL-1, IL-3, IL-9, IL-11, IL-12, IL-14, IL-17, IL-20, IL-21, granulocyte colony-stimulating factor (G-CSF), leukemia inhibitory factor (LIF), oncostatin M (OSM), CD 154, lymphotoxin (LT) beta, 4-IBB ligand (4-1BBL), a proliferation-inducing ligand (APRIL), CD70, CD153, CD178, glucocorticoid-induced TNFR-related ligand (GITRL), tumor necrosis factor superfamily member 14 (TNFSF14), OX40L, TNF- and ApoL-related leukocyte-expressed ligand 1 (TALL-1), or TNF-related apoptosis-inducing ligand (TRAIL).

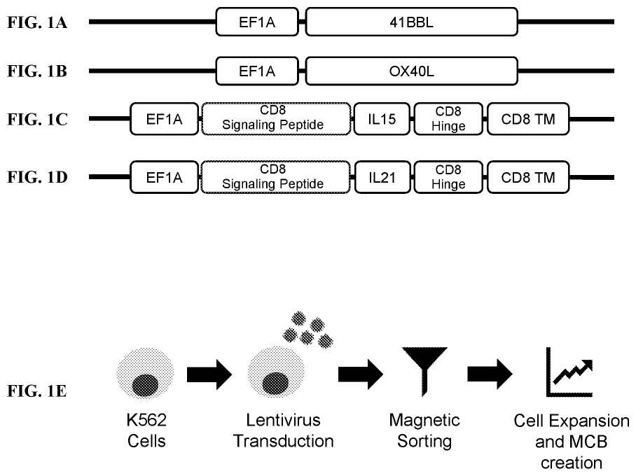

The term “engineered Autologous Cell Therapy,” which can be abbreviated as “eACT™,” also known as adoptive cell transfer, is a process by which a patient's own immune cells, e.g., T cells and/or NK cells, are collected and subsequently genetically altered to recognize and target one or more antigens expressed on the cell surface of one or more specific tumor cells or malignancies. Immune cells, e.g., T cells and/or NK cells, can be engineered to express, for example, chimeric antigen receptors (CAR) or T cell receptor (TCR). CAR positive (+) immune cells, e.g. T cells or immune cells, are engineered to express an extracellular single chain variable fragment (scFv) with specificity for a particular tumor antigen linked to an intracellular signaling part comprising a costimulatory domain and an activating domain. The costimulatory domain can be derived from, e.g., CD28, and the activating domain can be derived from, e.g., CD3-zeta (FIG. 1). In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Patent Publication Nos. 2013/0287748, 2014/0227237, 2014/0099309, and 2014/0050708, and these references are incorporated by reference in their entirety.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some embodiments, the immunomodulator targets a molecule on the surface of an immune cell, e.g., a T cell and/or an NK cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response.

“Immuno stimulating therapy” or “immuno stimulatory therapy” refers to a therapy that results in increasing (inducing or enhancing) an immune response in a subject for, e.g., treating cancer.

“Loaded” with a peptide, as used herein, refers to presentation of an antigen in the context of an MHC molecule. “Loaded” as used herein also means the binding of an antibody to an Fc binding receptor on a cell, such as CD32 and/or CD64.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses by inducing apoptosis or programmed cell death in the target cell. They were termed “natural killers” because NK cells do not require activation in order to kill a target cell. T-cells play a major role in cell-mediated-immunity. T-cell receptors (TCR) expressed on the surface of T cells differentiate T cells from other lymphocyte types. The thymus, a specialized organ of the immune system, is primarily responsible for T cell maturation. There are six types of T-cells, namely: Helper T-cells (e.g. CD4+ cells); Cytotoxic T-cells (also known as TC, cytotoxic T lymphocyte, CTL, T-killer cell, cytolytic T cell, CD8+ T-cells or killer T cell); Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2R.p, CXCR3, and LFA-1, and show numerous functional attributes distinctive of memory cells); (ii) central memory TCM cells express L-selectin and the CCR7, they secrete IL-2, but not IFNy or IL-4, and (iii) effector memory TEM cells, however, do not express L-selectin or CCR7 but produce effector cytokines like IFNy and IL-4); Regulatory T-cells (Tregs, suppressor T cells, or CD4+CD25+ regulatory T cells); Natural Killer T-cells (NKT); and Gamma Delta T-cells.

B-cells play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

The term “recombinant” cell, e.g., recombinant antigen-presenting cell (or “RAPC”), as used herein, is intended to refer to a cell, e.g., an antigen-presenting cell, that comprises a nucleic acid that is not naturally present in the cell, and can be a cell into which a recombinant expression vector has been introduced. It should be understood that such terms are intended to refer not only to the particular subject cell but also to the progeny of such a cell. Though certain modifications can occur in succeeding generations due to either mutation or environmental influences, such progeny are still included within the scope of the term “recombinant” as used herein. In some aspects, the RAPC is a bead-based artificial antigen presenting cell, an artificial lipid vesicle or other lipid bilayer containing system, an exosome, a solid support, or any combination thereof. Non-limiting examples of a solid support include a bead, a spherical or non-spherical nanoparticle support, a carbon nanotube support, a magnetic particle support, a filamentous polymer support, a two dimensional support or any combination thereof. Where the RAPC comprises a bead-based artificial antigen presenting cell, an artificial lipid vesicle or other lipid bilayer containing system, an exosome, a solid support, or any combination thereof, the RAPC can comprise one or more recombinant protein (e.g., one or more of an IL-21, an IL-15, a 4-1BBL, and/or an OX40L disclosed herein) associated with the surface of the RAPC.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on an immune cell, e.g., a T cell and/or an NK cell, thereby mediating a primary response by the immune cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on an immune cell, e.g., a T cell or an NK cell, that specifically binds with a cognate stimulatory ligand present on an antigen presenting cell (e.g., an aAPC of the invention, among others).

As used herein, the terms “subject” and “patient” are used interchangeably and refer to either a human or a non-human, such as primates, mammals, and vertebrates. In particular aspects, the subject is a human.

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs; full-length TCRs and antigen binding portions thereof; chimeric TCRs; TCR fusion constructs; and synthetic TCRs. In human, TCRs are expressed on the surface of T cells, and they are responsible for T cell recognition and targeting of antigen presenting cells. Antigen presenting cells (APCs) display fragments of foreign proteins (antigens) complexed with the major histocompatibility complex (MHC; also referred to herein as complexed with an HLA molecule, e.g., an HLA class 1 molecule). A TCR recognizes and binds to the antigen-HLA complex and recruits CD3 (expressed by T cells), activating the TCR. The activated TCR initiates downstream signaling and an immune response, including the destruction of the antigen-presenting cell.

In general, a TCR can comprise two chains, (i) an alpha chain and a beta chain for alpha-beta T cells, or (ii) a gamma chain and a delta chain for gamma-delta T cells, interconnected by disulfide bonds. Each chain comprises a variable domain (alpha chain variable domain, beta chain variable domain, gamma chain variable domain, and delta chain variable domain) and a constant region (alpha chain constant region, beta chain constant region, gamma chain constant region, and delta chain constant region). The variable domain is located distal to the cell membrane, and the variable domain interacts with an antigen. The constant region is located proximal to the cell membrane. A TCR can further comprises a transmembrane region and a short cytoplasmic tail. As used herein, the term “constant region” encompasses the transmembrane region and the cytoplasmic tail, when present, as well as the traditional “constant region.”

The variable domains can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, and FR4. Each variable domain contains a binding domain that interacts with an antigen. Though all three CDRs on each chain are involved in antigen binding, CDR3 is believed to be the primary antigen binding region. CDR1 is also interacts with the antigen, while CD2 is believed to primarily recognize the ELLA complex.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD3, as described herein), or a TCR described herein that is free from a cell membrane (e.g., an isolated TCR or a soluble TCR).

A “TCR fragment,” “antigen binding molecule,” or “portion of a TCR” refers to any portion of a TCR less than the whole. An antigen binding molecule can include the antigenic complementarity determining regions (CDRs).

The term “therapeutic benefit” or “therapeutically effective,” as used herein, refers to anything that promotes or enhances the well-being of the subject with respect to the medical treatment of this condition. This includes, but is not limited to, a reduction in the frequency or severity of the signs or symptoms of a disease.

As used herein, the term “treating” or “treatment” of a disease or condition refers to executing a protocol, which may include administering one or more therapy to a patient, in an effort to alleviate signs or symptoms of the disease. In some aspects, a treatment decreases the rate of disease progression, ameliorates or palliates the disease state, and/or facilitates remission or improved prognosis. Alleviation can occur prior to signs or symptoms of the disease or condition appearing, as well as after their appearance. Thus, in some aspects, “treating” or “treatment” includes “preventing” or “prevention” of a disease or an undesirable condition. However, “treating” or “treatment” does not require complete alleviation of all signs and/or symptoms, does not require a cure, and specifically includes protocols that have only a marginal effect on the patient.

As used herein, the terms “ug” and “uM” are used interchangeably with “μg” and “μM,” respectively.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. As described herein, any concentration range, percentage range, ratio range or integer range is to be understood to include the value of any integer within the recited range and, when appropriate, fractions thereof (such as one-tenth and one-hundredth of an integer), unless otherwise indicated.

Abbreviations used herein are defined throughout the present disclosure. Various aspects of the disclosure are described in further detail in the following subsections.

Various aspects described herein are described in further detail in the following subsections.

### II. Compositions of the Disclosure

Some aspects of the present disclosure are directed to recombinant antigen-presenting cells (RAPCs). In some aspects, the RAPC comprises (i) one or more one or more nucleic acid molecules encoding IL-21 and (ii) one or more nucleic acid molecules encoding OX40L. In some aspects, the RAPC comprises (i) one or more one or more nucleic acid molecules encoding IL-21, (ii) one or more nucleic acid molecules encoding OX40L, and (iii) one or more nucleic acid molecule encoding 4-1BBL. In some aspects, the RAPC comprises (i) one or more one or more nucleic acid molecules encoding IL-21, (ii) one or more nucleic acid molecules encoding OX40L, and (iii) one or more nucleic acid molecules encoding IL-15. In some aspects, the RAPC comprises (i) one or more one or more nucleic acid molecules encoding IL-21, (ii) one or more nucleic acid molecules encoding OX40L, (iii) one or more nucleic acid molecule encoding 4-1BBL, and (iv) one or more nucleic acid molecules encoding IL-15.

Some aspects of the present disclosure are directed to a population of RAPCs, comprising a first RAPC and a second RAPC. In some aspects, the first RAPC and the second RAPC comprise (i) one or more nucleic acid molecules encoding IL-21 and (ii) one or more nucleic acid molecules encoding OX40L. In some aspects, the first RAPC and the second RAPC comprise (i) one or more nucleic acid molecules encoding IL-21, (ii) one or more nucleic acid molecules encoding OX40L, and (iii) one or more nucleic acid molecules encoding 4-1BBL. In some aspects, the first RAPC and the second RAPC comprise (i) one or more nucleic acid molecules encoding IL-21, (ii) one or more nucleic acid molecules encoding OX40L, and (iii) one or more nucleic acid molecules encoding IL-15. In some aspects, the first RAPC and the second RAPC comprise (i) one or more nucleic acid molecules encoding IL-21, (ii) one or more nucleic acid molecules encoding OX40L, (iii) one or more nucleic acid molecules encoding 4-1BBL, and (iv) one or more nucleic acid molecules encoding IL-15.

In some aspects, (a) the first RAPC comprises one or more nucleic acid molecules encoding IL-21 and (b) the second RAPC comprises one or more nucleic acid molecules encoding OX40L.

In some aspects, (a) the first RAPC comprises (i) one or more nucleic acid molecules encoding IL-21 and (ii) one or more nucleic acid molecules encoding OX40L; and (b) the second RAPC comprises one or more nucleic acid molecules encoding 4-1BBL. In some aspects, (a) the first RAPC comprises (i) one or more nucleic acid molecules encoding IL-21 and (ii) one or more nucleic acid molecules encoding 4-1BBL; and (b) the second RAPC comprises one or more nucleic acid molecules encoding OX40L. In some aspects, (a) the first RAPC comprises (i) one or more nucleic acid molecules encoding 4-1BBL and (ii) one or more nucleic acid molecules encoding OX40L; and (b) the second RAPC comprises one or more nucleic acid molecules encoding IL-21.

In some aspects, (a) the first RAPC comprises (i) one or more nucleic acid molecules encoding IL-21 and (ii) one or more nucleic acid molecules encoding OX40L; and (b) the second RAPC comprises one or more nucleic acid molecules encoding IL-15. In some aspects, (a) the first RAPC comprises (i) one or more nucleic acid molecules encoding IL-21 and (ii) one or more nucleic acid molecules encoding IL-15; and (b) the second RAPC comprises one or more nucleic acid molecules encoding OX40L. In some aspects, (a) the first RAPC comprises (i) one or more nucleic acid molecules encoding IL-15 and (ii) one or more nucleic acid molecules encoding OX40L; and (b) the second RAPC comprises one or more nucleic acid molecules encoding IL-21.

In some aspects, (a) the first RAPC comprises (i) one or more nucleic acid molecules encoding IL-21 and (ii) one or more nucleic acid molecules encoding OX40L; and (b) the second RAPC comprises (i) one or more nucleic acid molecules encoding 4-1BBL and (ii) one or more nucleic acid molecules encoding IL-15. In some aspects, (a) the first RAPC comprises (i) one or more nucleic acid molecules encoding IL-21 and (ii) one or more nucleic acid molecules encoding 4-1BBL; and (b) the second RAPC comprises (i) one or more nucleic acid molecules encoding OX40L and (ii) one or more nucleic acid molecules encoding IL-15. In some aspects, (a) the first RAPC comprises (i) one or more nucleic acid molecules encoding IL-21 and (ii) one or more nucleic acid molecules encoding IL-15; and (b) the second RAPC comprises (i) one or more nucleic acid molecules encoding 4-1BBL and (ii) one or more nucleic acid molecules encoding OX40L.

In some aspects, (a) the first RAPC comprises (i) one or more nucleic acid molecules encoding IL-21, (ii) one or more nucleic acid molecules encoding OX40L, and (iii) one or more nucleic acid molecules encoding 4-1BBL; and (b) the second RAPC comprises one or more nucleic acid molecules encoding IL-15. In some aspects, (a) the first RAPC comprises (i) one or more nucleic acid molecules encoding IL-21, (ii) one or more nucleic acid molecules encoding OX40L, and (iii) one or more nucleic acid molecules encoding IL-15; and (b) the second RAPC comprises one or more nucleic acid molecules encoding 4-1BBL. In some aspects, (a) the first RAPC comprises (i) one or more nucleic acid molecules encoding 4-1BBL, (ii) one or more nucleic acid molecules encoding OX40L, and (iii) one or more nucleic acid molecules encoding IL-15; and (b) the second RAPC comprises one or more nucleic acid molecules encoding IL-21. In some aspects, (a) the first RAPC comprises (i) one or more nucleic acid molecules encoding IL-21, (ii) one or more nucleic acid molecules encoding 4-1BBL, and (iii) one or more nucleic acid molecules encoding IL-15; and (b) the second RAPC comprises one or more nucleic acid molecules encoding OX40L.

In some aspects, the population of RAPCs comprises a third RAPC, wherein the third RAPC comprises (i) one or more nucleic acid molecules encoding IL-21, (ii) one or more nucleic acid molecules encoding OX40L, (iii) one or more nucleic acid molecules encoding 4-1BBL, (iv) one or more nucleic acid molecules encoding IL-15, or (v) any combination of (i)-(iv). In some aspects, the population of RAPCs comprises a fourth RAPC, wherein the fourth RAPC comprises (i) one or more nucleic acid molecules encoding IL-21, (ii) one or more nucleic acid molecules encoding OX40L, (iii) one or more nucleic acid molecules encoding 4-1BBL, (iv) one or more nucleic acid molecules encoding IL-15, or (v) any combination of (i)-(iv). In some aspects, the population of RAPCs comprises a first RAPC, a second RAPC, a third RAPC, and a fourth RAPC; wherein (a) the first RAPC comprises one or more nucleic acid molecules encoding IL-21, (b) the second RAPC expresses one or more nucleic acid molecules encoding OX40L, (c) the third RAPC comprises one or more nucleic acid molecules encoding 4-1BBL, and (d) the fourth RAPC comprises one or more nucleic acid molecules encoding IL-15.

In some aspects, the first RAPC and the second RAPC comprise (i) one or more nucleic acid molecules encoding IL-21, (ii) one or more nucleic acid molecules encoding OX40L, and (iii) one or more nucleic acid molecules encoding 4-1BBL. In some aspects, the first RAPC and the second RAPC comprise (i) one or more nucleic acid molecules encoding IL-21, (ii) one or more nucleic acid molecules encoding OX40L, and (iii) one or more nucleic acid molecules encoding IL-15. In some aspects, the first RAPC and the second RAPC comprise (i) one or more nucleic acid molecules encoding IL-21, (ii) one or more nucleic acid molecules encoding OX40L, (iii) one or more nucleic acid molecules encoding 4-1BBL, and (iv) one or more nucleic acid molecules encoding IL-15.

In some aspects, the one or more one or more nucleic acid molecules encoding IL-21 are heterologous to the cell. In some aspects, the one or more one or more nucleic acid molecules encoding OX40L are heterologous to the cell. In some aspects, the one or more one or more nucleic acid molecules encoding 4-1BBL are heterologous to the cell. In some aspects, the one or more one or more nucleic acid molecules encoding IL-15 are heterologous to the cell.

In some aspects, the RAPC expresses one or more of IL-21, OX40L, 4-1BBL, and IL-15. In some aspects, the RAPC expresses IL-21 and OX40L. In some aspects, the RAPC expresses IL-21, OX40L, and 4-1BBL. In some aspects, the RAPC expresses IL-21, OX40L, and IL-15. In some aspects, the RAPC expresses IL-21, OX40L, 4-1BBL, and IL-15.

In some aspects, one or more of IL-21, OX40L, 4-1BBL, and IL-15 are expressed on the surface of the RAPC. In some aspects, IL-21 and OX40L are expressed on the surface of the RAPC. In some aspects, IL-21, OX40L, and 4-1BBL are expressed on the surface of the RAPC. In some aspects, IL-21, OX40L, and IL-15 are expressed on the surface of the RAPC. In some aspects, IL-21, OX40L, 4-1BBL, and IL-15 are expressed on the surface of the RAPC.

### II.A. Recombinant Antigen-Presenting Cells

Certain aspects of the present disclosure are directed to recombinant antigen-presenting cells (RAPCs). Any antigen-presenting cell (APC) can be used in the compositions and methods disclosed herein. In some aspects, the APC comprises a cell, e.g., a mammalian cell, e.g., a human cell. In some aspects, the cell is a tumor cell. In some aspects, the cell is derived from a tumor cell. In some aspects, the cell is a K562 cell. In some aspects, the cell is a genetically modified K562 cell. The K562 cell line is stable cell line derived from a bone marrow sample obtained from a human patient afflicted with chronic myelogenous leukemia (CML). K562 cells ae culture in suspension, and they have a lymphoblast morphology.

In certain aspects, the RAPC comprises a K562 cell, comprising (i) one or more nucleic acid molecules encoding IL-21 and (ii) one or more nucleic acid molecules encoding OX40L. In some aspects, the RAPC comprises a K562 cell, comprising (i) one or more nucleic acid molecules encoding IL-21, (ii) one or more nucleic acid molecules encoding OX40L, and (iii) one or more nucleic acid molecules encoding 4-1BBL. In some aspects, the RAPC comprises a K562 cell, comprising (i) one or more nucleic acid molecules encoding IL-21, (ii) one or more nucleic acid molecules encoding OX40L, and (iii) one or more nucleic acid molecules encoding IL-15. In some aspects, the RAPC comprises a K562 cell, comprising (i) one or more nucleic acid molecules encoding IL-21, (ii) one or more nucleic acid molecules encoding OX40L, (iii) one or more nucleic acid molecules encoding 4-1BBL, and (iv) one or more nucleic acid molecules encoding IL-15.

In some aspects, a population of RAPCs disclosed herein comprises a first RAPC, a second RAPC, a third RAPC, and a fourth RAPC; wherein the first RAPC, the second RAPC, the third RAPC, the fourth RAPC, or any combination thereof is a K562 cell. In some aspects, a population of RAPCs disclosed herein comprises a first RAPC, a second RAPC, a third RAPC, and a fourth RAPC; wherein the first RAPC, the second RAPC, the third RAPC, the fourth RAPC, or any combination thereof is a modified K562 cell.

In some aspects, the APC comprises an immune cell, e.g., a mammalian immune cell, e.g., a human immune cell. Any immune cell capable of presenting an antigen can be used in the compositions and methods disclosed herein. In some aspects, the immune cell comprises a dendritic cell. In some aspects, the cell is an engineered clonal cell.

In some aspects, the APC is a cell-like antigen-presenting surface that expresses one or more antigen. In some aspects, the APC comprises a bead-based artificial antigen presenting cell. In some aspects, the APC comprises a latex bead comprising one or more of IL-21, OX40L, 4-1BBL, and IL-15 on its surface. In some aspects, the APC comprises a polystyrene bead comprising one or more of IL-21, OX40L, 4-1BBL, and IL-15 on its surface. In some aspects, the APC comprises a metal bead comprising one or more of IL-21, OX40L, 4-1BBL, and IL-15 on its surface.

In some aspects, the APC comprises an artificial lipid vesicle or other lipid bilayer containing system. In some aspects, the APC comprises an endosome. In some aspects, the APC comprises an exosome. In some aspects, the APC comprises a microvesicle. In some aspects, the APC comprises a nanovesicle. In some aspects, the APC comprises a liposome. In some aspects, the APC comprises a solid support. In some aspects, the APC comprises a bead. In some aspects, the APC comprises a spherical or non-spherical nanoparticle support. In some aspects, the APC comprises a carbon nanotube support. In some aspects, the APC comprises a magnetic particle support. In some aspects, the APC comprises a filamentous polymer support. In some aspects, the APC comprises a two dimensional support.

The RAPCs disclosed herein can be generated using any methods. In some aspects, RAPCs are generated by genetically engineering an APC to comprise one or more of the nucleic acid molecules disclosed herein, e.g., encoding IL-21, IL-15, 4-1BBL, and/or OX40L. In some aspects, K562 cells are engineered by lentiviral transduction, e.g., with lentiviral vectors comprising nucleic acid molecules encoding IL-21 (e.g., an IL-21-Fc or IL-21-CD8 fusion construct disclosed herein), IL-15 (e.g., an IL-15-Fc or IL-15-CD8 fusion construct disclosed herein), 4-1BBL, and/or OX40L.

In some aspects, the APCs are irradiated. Irradiation of APCs has been shown to improve the properties of immune cells cultured on the irradiated APCs, including increasing expression of CD70 in APCs, which is thought to facilitate activation and/or expansion of immune cells. See e.g., Huang et al., J. Immunother. 34(4):327 (2011), which is incorporated by reference herein in its entirety. In some aspects, immune cells cultured in the presence of irradiated APCs, e.g., RAPCs, have increased proliferation and/or increased IFNg production as compared to immune cells cultured in the presence of non-irradiated APCs, e.g., RAPCs. In some aspects, the APCs, e.g., the RAPCs, are irradiated to render them incapable of growth or division. In some aspects, the APCs, e.g., RAPCs, are irradiated at least about 10 Gy, at least about 20 Gy, at least about 30 Gy, at least about 40 Gy, at least about 50 Gy, at least about 60 Gy, at least about 70 Gy, at least about 80 Gy, at least about 90 Gy, at least about 100 Gy, at least about 125 Gy, at least about 150 Gy, at least about 175 Gy, or at least about 200 Gy. In some aspects, the APCs, e.g., RAPCs, are irradiated at about 100 Gy. In some aspects, the APCs, e.g., RAPCs, are contacted with Mitomycin C. In some aspects, the APCs, e.g., RAPCs, are contacted with at least about 25 μg/mL Mitomycin C.

In some aspects, an antigen-presenting cell described herein is a 721.221 cell. In some aspects, the antigen-presenting cell described herein is a 721.221 cell, which is modified to express one or more of IL-21, OX40L, 4-1BBL, and IL-15. In some aspects, antigen-presenting cell described herein is a 721.221 cell, which is modified to express IL-21, OX40L, 4-1BBL, and IL-15. In some aspects, an antigen-presenting cell described herein is a Hut78 cell. In some aspects, the antigen-presenting cell described herein is a Hut78 cell, which is modified to express one or more of IL-21, OX40L, 4-1BBL, and IL-15. In some aspects, antigen-presenting cell described herein is a Hut78 cell, which is modified to express IL-21, OX40L, 4-1BBL, and IL-15.

### II.B. IL-21

Some aspects of the present disclosure are directed to RAPCs comprising one or more nucleic acid molecules encoding IL-21. As used herein, IL-21 refers to interleukin-21, functional fragments thereof, functional variants thereof, or fusion polypeptides comprising IL-21 or a functional fragment or functional variant thereof. In some aspects, the IL-21 is any polypeptide that has at least one function of wild-type IL-21. In some aspects, the IL-21 comprises a mammalian IL-21. In some aspects, the IL-21 comprises a human IL-21 (UniProtKB-Q9HBE4).

Human IL-21 is a cytokine with immunoregulatory activity, which is believed to promote the transition between innate and adaptive immunity. IL-21 induces the production of IgG1 and IgG3 in B-cells, and has been implicated in the generation and maintenance of T follicular helper (Tfh) cells and the formation of germinal-centers. Together with IL-6, IL-21 controls the early generation of Tfh cells and is critical for an effective antibody response to acute viral infection. IL-21 may also play a role in proliferation and maturation of natural killer (NK) cells in synergy with IL-15, and may regulate proliferation of mature B- and T-cells in response to activating stimuli. In synergy with IL-15 and IL-18, IL-21 stimulates interferon gamma production in T-cells and NK cells. IL-21 may also inhibit dendritic cells (DC) activation and maturation during T-cell mediated immune response. The canonical sequence is provided in Table 1A.

TABLE 1A
Human IL-21 Canonical Amino Acid and
Nucleic Acid Sequences.

MRSSPGNMERIVICLMVIFLGTLVHKSSSQGQDRHMIRMRQLIDIVDQL
KNYVNDLVPEFLPAPEDVETNCEWSAFSCFQKAQLKSANTGNNERIINV
SIKKLKRKPPSTNAGRRQKHRLTCPSCDSYEKKPPKEFLERFKSLLQKM
IHQHLSSRTHGSEDS
(SEQ ID NO: 1)
ATGCGCAGCAGCCCGGGCAACATGGAACGCATTGTGATTTGCCTGATGG
TGATTTTTCTGGGCACCCTGGTGCATAAAAGCAGCAGCCAGGGCCAGGA
TCGCCATATGATTCGCATGCGCCAGCTGATTGATATTGTGGATCAGCTG
AAAAACTATGTGAACGATCTGGTGCCGGAATTTCTGCCGGCGCCGGAAG
ATGTGGAAACCAACTGCGAATGGAGCGCGTTTAGCTGCTTTCAGAAAGC
GCAGCTGAAAAGCGCGAACACCGGCAACAACGAACGCATTATTAACGTG
AGCATTAAAAAACTGAAACGCAAACCGCCGAGCACCAACGCGGGCCGCC
GCCAGAAACATCGCCTGACCTGCCCGAGCTGCGATAGCTATGAAAAAAA
ACCGCCGAAAGAATTTCTGGAACGCTTTAAAAGCCTGCTGCAGAAAATG
ATTCATCAGCATCTGAGCAGCCGCACCCATGGCAGCGAAGATAGC
(SEQ ID NO: 5)

In some aspects, the nucleic acid molecule encoding IL-21 encodes a polypeptide comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 1, wherein the polypeptide has an IL-21 activity. In some aspects, the nucleic acid molecule encoding IL-21 encodes a polypeptide comprising the amino acid sequence set forth in SEQ ID NO: 1.

In some aspects, the nucleic acid molecule encoding IL-21 comprises a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 5, wherein the nucleic acid molecule encodes a polypeptide that has an IL-21 activity. In some aspects, the nucleic acid molecule encoding IL-21 comprises the nucleic acid sequence set forth in SEQ ID NO: 5.

In some aspects, the IL-21 is a fusion protein. In some aspects, the IL-21 is a fusion protein comprising a human IL-21 polypeptide and an Fc region of an immunoglobulin. In some aspects, the IL-21 comprises (i) a human IL-21 polypeptide, (ii) the Fc region of an immunoglobulin, and (iii) a transmembrane domain. In some aspects, the IL-21 comprises (i) a human IL-21 polypeptide, (ii) a hinge region, (iii) the Fc region of an immunoglobulin, and (iv) a transmembrane domain. In some aspects, the IL-21 comprises (i) a human IL-21 polypeptide, (ii) a hinge region, (iii) the Fc region of an immunoglobulin, (iv) a transmembrane domain, and (v) a signaling peptide.

Any Fc region can be used in the IL-21 fusion polypeptides disclosed herein. In some aspects, the Fc region is an IgG Fc region, an IgA Fc region, or an IgD Fc region. In some aspects, the Fc region is an IgG1 Fc region, an IgG2 Fc region, and IgG3 Fc region, an IgG4 Fc region, or a combination thereof. In some aspects, the IL-21 fusion protein comprises an IgG1 Fc region. In some aspects, the IL-21 fusion protein comprises an IgG4 Fc region.

Any hinge region can be used in the IL-21 fusion polypeptides disclosed herein. In some aspects, the hinge region comprises an Fc hinge region. In some aspects, the hinge region comprises an IgG Fc hinge region, an IgA Fc hinge region, or an IgD Fc hinge region. In some aspects, the hinge region comprises an IgG1 Fc hinge region, an IgG2 Fc hinge region, and IgG3 Fc hinge region, an IgG4 hinge Fc region, or a combination thereof. In some aspects, the IL-21 fusion protein comprises an IgG1 Fc hinge region. In some aspects, the IL-21 fusion protein comprises an IgG4 Fc hinge region. In some aspects, the IL-21 fusion protein comprises a modified IgG4 Fc hinge region. In some aspects, the hinge region comprises a CD8 hinge region.

Any transmembrane domain can be used in the IL-21 fusion polypeptides disclosed herein. In some aspects the transmembrane domain comprises a CD4 transmembrane domain. In some aspects the transmembrane domain comprises a CD8 transmembrane domain.

Any signaling peptide can be used in the IL-21 fusion polypeptides disclosed herein. In some aspects the signaling peptide comprises a GMSCF signaling peptide. In some aspects the signaling peptide comprises a GMSCFR signaling peptide. In some aspects, the signaling peptide comprises a CD8 signaling peptide.

In some aspects, the IL-21 comprises (i) a CD8 signaling peptide, (ii) a human IL-21 polypeptide, (iii) a CD8 hinge region, and (iii) a CD8 transmembrane domain.

### II.C. IL-15

Some aspects of the present disclosure are directed to RAPCs comprising one or more nucleic acid molecules encoding IL-15. As used herein, IL-15 refers to interleukin-15, functional fragments thereof, functional variants thereof, or fusion polypeptides comprising IL-15 or a functional fragment or functional variant thereof. In some aspects, the IL-15 is any polypeptide that has at least one function of wild-type IL-15. In some aspects, the IL-15 comprises a mammalian IL-15. In some aspects, the IL-15 comprises a human IL-15 (UniProtKB-P40933).

Human IL-15 is a cytokine that stimulates the proliferation of T-lymphocytes. Stimulation by IL-15 requires interaction of IL-15 with components of the IL-2 receptor, including IL-2RB and likely IL-2RG but not IL-2RA. In neutrophils, IL-15 stimulates phagocytosis likely by signaling through the IL-15 receptor, composed of the subunits IL-15RA, IL-2RB, and IL-2RG, which results in kinase SYK activation. The canonical sequence is provided in Table 1B.

TABLE 1B
Human IL-15 Canonical Amino Acid and
Nucleic Acid Sequences.

MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS
(SEQ ID NO: 2)
ATGCGCATTAGCAAACCGCATCTGCGCAGCATTAGCATTCAGTGCTATC
TGTGCCTGCTGCTGAACAGCCATTTTCTGACCGAAGCGGGCATTCATGT
GTTTATTCTGGGCTGCTTTAGCGCGGGCCTGCCGAAAACCGAAGCGAAC
TGGGTGAACGTGATTAGCGATCTGAAAAAAATTGAAGATCTGATTCAGA
GCATGCATATTGATGCGACCCTGTATACCGAAAGCGATGTGCATCCGAG
CTGCAAAGTGACCGCGATGAAATGCTTTCTGCTGGAACTGCAGGTGATT
AGCCTGGAAAGCGGCGATGCGAGCATTCATGATACCGTGGAAAACCTGA
TTATTCTGGCGAACAACAGCCTGAGCAGCAACGGCAACGTGACCGAAAG
CGGCTGCAAAGAATGCGAAGAACTGGAAGAAAAAAACATTAAAGAATTT
CTGCAGAGCTTTGTGCATATTGTGCAGATGTTTATTAACACCAGC
(SEQ ID NO: 6)

In some aspects, the nucleic acid molecule encoding IL-15 encodes a polypeptide comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 2, wherein the polypeptide has an IL-15 activity. In some aspects, the nucleic acid molecule encoding IL-15 encodes a polypeptide comprising the amino acid sequence set forth in SEQ ID NO: 2.

In some aspects, the nucleic acid molecule encoding IL-15 comprises a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 6, wherein the nucleic acid molecule encodes a polypeptide that has an IL-15 activity. In some aspects, the nucleic acid molecule encoding IL-15 comprises the nucleic acid sequence set forth in SEQ ID NO: 6.

In some aspects, the IL-15 is a fusion protein. In some aspects, the IL-15 is a fusion protein comprising a human IL-15 polypeptide and an Fc region of an immunoglobulin. In some aspects, the IL-15 comprises (i) a human IL-15 polypeptide, (ii) the Fc region of an immunoglobulin, and (iii) a transmembrane domain. In some aspects, the IL-15 comprises (i) a human IL-15 polypeptide, (ii) a hinge region, (iii) the Fc region of an immunoglobulin, and (iv) a transmembrane domain. In some aspects, the IL-15 comprises (i) a human IL-15 polypeptide, (ii) a hinge region, (iii) the Fc region of an immunoglobulin, (iv) a transmembrane domain, and (v) a signaling peptide.

Any Fc region can be used in the IL-15 fusion polypeptides disclosed herein. In some aspects, the Fc region is an IgG Fc region, an IgA Fc region, or an IgD Fc region. In some aspects, the Fc region is an IgG1 Fc region, an IgG2 Fc region, and IgG3 Fc region, an IgG4 Fc region, or a combination thereof. In some aspects, the IL-15 fusion protein comprises an IgG1 Fc region. In some aspects, the IL-15 fusion protein comprises an IgG4 Fc region.

Any hinge region can be used in the IL-15 fusion polypeptides disclosed herein. In some aspects, the hinge region comprises an Fc hinge region. In some aspects, the hinge region comprises an IgG Fc hinge region, an IgA Fc hinge region, or an IgD Fc hinge region. In some aspects, the hinge region comprises an IgG1 Fc hinge region, an IgG2 Fc hinge region, and IgG3 Fc hinge region, an IgG4 hinge Fc region, or a combination thereof. In some aspects, the IL-15 fusion protein comprises an IgG1 Fc hinge region. In some aspects, the IL-15 fusion protein comprises an IgG4 Fc hinge region. In some aspects, the IL-15 fusion protein comprises a modified IgG4 Fc hinge region. In some aspects, the hinge region comprises a CD8 hinge region.

Any transmembrane domain can be used in the IL-15 fusion polypeptides disclosed herein. In some aspects the transmembrane domain comprises a CD4 transmembrane domain. In some aspects the transmembrane domain comprises a CD8 transmembrane domain.

Any signaling peptide can be used in the IL-15 fusion polypeptides disclosed herein. In some aspects the signaling peptide comprises a GMSCF signaling peptide. In some aspects the signaling peptide comprises a GMSCFR signaling peptide. In some aspects, the signaling peptide comprises a CD8 signaling peptide.

In some aspects, the IL-15 comprises (i) a CD8 signaling peptide, (ii) a human IL-15 polypeptide, (iii) a CD8 hinge region, and (iii) a CD8 transmembrane domain.

### II.D. 4-1BBL

Some aspects of the present disclosure are directed to RAPCs comprising one or more nucleic acid molecules encoding 4-1BBL. 4-1BBL is also known as tumor necrosis factor ligand superfamily member 9 (or TNFSF9). As used herein, 4-1BBL refers to full length 4-1BBL, functional fragments thereof, functional variants thereof, or fusion polypeptides comprising 4-1BBL or a functional fragment or functional variant thereof. In some aspects, the 4-1BBL is any polypeptide that has at least one function of wild-type 4-1BBL. In some aspects, the 4-1BBL comprises a mammalian 4-1BBL. In some aspects, the 4-1BBL comprises a human 4-1BBL (UniProtKB-P41273).

Human 4-1BB is a ligand that binds to TNFRSF9 (4-1BBL) receptor. This interaction induces the proliferation of activated peripheral blood T-cells. 4-1BBL further plays a role in activation-induced cell death (AICD) as well as in cognate interactions between T-cells and B-cells/macrophages. The canonical sequence is provided in Table 1C.

TABLE 1C
Human 4-1BBL Canonical Amino Acid and
Nucleic Acid Sequences.

MEYASDASLDPEAPWPPAPRARACRVLPWALVAGLLLLLLLAAACAVFL
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE
(SEQ ID NO: 3)
ATGGAATATGCGAGCGATGCGAGCCTGGATCCGGAAGCGCCGTGGCCGC
CGGCGCCGCGCGCGCGCGCGTGCCGCGTGCTGCCGTGGGCGCTGGTGGC
GGGCCTGCTGCTGCTGCTGCTGCTGGCGGCGGCGTGCGCGGTGTTTCTG
GCGTGCCCGTGGGCGGTGAGCGGCGCGCGCGCGAGCCCGGGCAGCGCGG
CGAGCCCGCGCCTGCGCGAAGGCCCGGAACTGAGCCCGGATGATCCGGC
GGGCCTGCTGGATCTGCGCCAGGGCATGTTTGCGCAGCTGGTGGCGCAG
AACGTGCTGCTGATTGATGGCCCGCTGAGCTGGTATAGCGATCCGGGCC
TGGCGGGCGTGAGCCTGACCGGCGGCCTGAGCTATAAAGAAGATACCAA
AGAACTGGTGGTGGCGAAAGCGGGCGTGTATTATGTGTTTTTTCAGCTG
GAACTGCGCCGCGTGGTGGCGGGCGAAGGCAGCGGCAGCGTGAGCCTGG
CGCTGCATCTGCAGCCGCTGCGCAGCGCGGCGGGCGCGGCGGCGCTGGC
GCTGACCGTGGATCTGCCGCCGGCGAGCAGCGAAGCGCGCAACAGCGCG
TTTGGCTTTCAGGGCCGCCTGCTGCATCTGAGCGCGGGCCAGCGCCTGG
GCGTGCATCTGCATACCGAAGCGCGCGCGCGCCATGCGTGGCAGCTGAC
CCAGGGCGCGACCGTGCTGGGCCTGTTTCGCGTGACCCCGGAAATTCCG
GCGGGCCTGCCGAGCCCGCGCAGCGAA
(SEQ ID NO: 7)

In some aspects, the nucleic acid molecule encoding 4-1BBL encodes a polypeptide comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 3, wherein the polypeptide has an 4-1BBL activity. In some aspects, the nucleic acid molecule encoding 4-1BBL encodes a polypeptide comprising the amino acid sequence set forth in SEQ ID NO: 3.

In some aspects, the nucleic acid molecule encoding 4-1BBL comprises a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 7, wherein the nucleic acid molecule encodes a polypeptide that has a 4-1BBL activity. In some aspects, the nucleic acid molecule encoding 4-1BBL comprises the nucleic acid sequence set forth in SEQ ID NO: 7.

### II.E. OX40L

Some aspects of the present disclosure are directed to RAPCs comprising one or more nucleic acid molecules encoding OX40L. OX40L is also known as tumor necrosis factor ligand superfamily member 4 (or TNFSF4). As used herein, OX40L refers to full length OX40L, functional fragments thereof, functional variants thereof, or fusion polypeptides comprising OX40L or a functional fragment or functional variant thereof. In some aspects, the OX40L is any polypeptide that has at least one function of wild-type OX40L. In some aspects, the OX40L comprises a mammalian OX40L. In some aspects, the OX40L comprises a human OX40L (UniProtKB-P23510).

Human OX40 is a ligand that binds to TNFRSF4 (OX40L) receptor. OX40L co-stimulates T-cell proliferation and cytokine production. The canonical sequence is provided in Table 1D.

TABLE 1D
Human OX40L Canonical Amino Acid and
Nucleic Acid Sequences.

MERVQPLEENVGNAARPRFERNKLLLVASVIQGLGLLLCFTYICLHFSA
LQVSHRYPRIQSIKVQFTEYKKEKGFILTSQKEDEIMKVQNNSVIINCD
GFYLISLKGYFSQEVNISLHYQKDEEPLFQLKKVRSVNSLMVASLTYKD
KVYLNVTTDNTSLDDFHVNGGELILIHQNPGEFCVL
(SEQ ID NO: 4)
ATGGAACGCGTGCAGCCGCTGGAAGAAAACGTGGGCAACGCGGCGCGCC
CGCGCTTTGAACGCAACAAACTGCTGCTGGTGGCGAGCGTGATTCAGGG
CCTGGGCCTGCTGCTGTGCTTTACCTATATTTGCCTGCATTTTAGCGCG
CTGCAGGTGAGCCATCGCTATCCGCGCATTCAGAGCATTAAAGTGCAGT
TTACCGAATATAAAAAAGAAAAAGGCTTTATTCTGACCAGCCAGAAAGA
AGATGAAATTATGAAAGTGCAGAACAACAGCGTGATTATTAACTGCGAT
GGCTTTTATCTGATTAGCCTGAAAGGCTATTTTAGCCAGGAAGTGAACA
TTAGCCTGCATTATCAGAAAGATGAAGAACCGCTGTTTCAGCTGAAAAA
AGTGCGCAGCGTGAACAGCCTGATGGTGGCGAGCCTGACCTATAAAGAT
AAAGTGTATCTGAACGTGACCACCGATAACACCAGCCTGGATGATTTTC
ATGTGAACGGCGGCGAACTGATTCTGATTCATCAGAACCCGGGCGAATT
TTGCGTGCTG
(SEQ ID NO: 8)

In some aspects, the nucleic acid molecule encoding OX40L encodes a polypeptide comprising an amino acid sequence having at least about 80%, at least about 85%, at least about 90%, at least about 95%, at least about 96%, at least about 97%, at least about 98%, or at least about 99% sequence identity to the amino acid sequence set forth in SEQ ID NO: 4, wherein the polypeptide has an OX-40L activity. In some aspects, the nucleic acid molecule encoding OX40L encodes a polypeptide comprising the amino acid sequence set forth in SEQ ID NO: 4.

In some aspects, the nucleic acid molecule encoding OX40L comprises a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 8, wherein the nucleic acid molecule encodes a polypeptide that has an OX40L activity. In some aspects, the nucleic acid molecule encoding OX40L comprises the nucleic acid sequence set forth in SEQ ID NO: 8.

### II.F. Other Nucleic Acid Molecules

In some aspects, the RAPCs comprise one or more additional nucleic acid molecules, wherein the one or more additional nucleic acid molecules encodes IL-2. In some aspects, the RAPCs further express IL-2.

In some aspects, the RAPCs express one or more co-stimulatory molecule. In some aspects, the co-stimulatory molecule comprises CD86, B7.1, or a fragment or variant thereof. In some aspects, the RAPCs comprise one or more nucleic acid molecules encoding CD86. In some aspects, the RAPCs further express CD86. In some aspects, the RAPCs comprise one or more nucleic acid molecules encoding B7.1. In some aspects, the RAPCs further express B7.1. In some aspects, the RAPCs comprise (i) one or more nucleic acid molecules encoding CD86 and (ii) one or more nucleic acid molecules encoding B7.1. In some aspects, the RAPCs express CD86 and B7.1.

In some aspects, the RAPCs express one or more SLAM family antigens. In some aspects, the SLAM family antigen is selected from CD2, CD48, CD58 (LFA-3), CD244 (2B4), CD229 (Ly9), CD319 (CS1 (CD2 subset 1); CRACC (CD2-like receptor activating cytotoxic cells)), and CD352 (NTB-A (NK-T-B antigen)). In some aspects, the RAPCs comprise one or more nucleic acid molecules encoding CD48. In some aspects, the RAPCs further express CD48. In some aspects, the RAPCs comprise one or more nucleic acid molecules encoding CS1 (CD319). In some aspects, the RAPCs further express CS1 (CD319). In some aspects, the RAPCs further express CD48 and CS1 (CD319).

In some aspects, the RAPCs comprise one or more nucleic acid molecule encoding an adhesion polypeptide. In some aspects, the adhesion polypeptide comprises ICAM-1 (CD54), LFA-3, or a fragment or variant thereof. In some aspects, the RAPCs comprise one or more nucleic acid molecules encoding ICAM-1 (CD54). In some aspects, the RAPCs further express ICAM-1 (CD54). In some aspects, the RAPCs comprise one or more nucleic acid molecules encoding LFA-3 (CD58). In some aspects, the RAPCs further express LFA-3 (CD58). In some aspects, the RAPCs comprise (i) one or more nucleic acid molecules encoding ICAM-1 (CD54) and (ii) one or more nucleic acid molecules encoding LFA-3 (CD58). In some aspects, the RAPCs express ICAM-1 (CD54) and LFA-3 (CD58).

In some aspects, the RAPCs do not express endogenous HLA class I molecules. In some aspects, the RAPCs do not express endogenous HLA class II molecules. In some aspects, the RAPCs do not express endogenous MHC CDId molecules. In some aspects, the RAPCs do not express endogenous HLA class I, II, and/or CD1d molecules.

### III. Methods of the Disclosure

Some aspects of the present disclosure are directed to methods of expanding a population of immune cells ex vivo. In some aspects, the method comprises contacting the population of immune cells with an RAPC disclosed herein. In some aspects, the RAPC expresses IL-21 (e.g., an IL-21-Fc or IL-21-CD8 fusion construct disclosed herein) and OX40L. In some aspects, the RAPC expresses IL-21 (e.g., IL-21-Fc or IL-21-CD8), OX40L, and IL-15 (e.g., an IL-15-Fc or IL-15-CD8 fusion construct disclosed herein). In some aspects, the RAPC expresses IL-21 (e.g., IL-21-Fc or IL-21-CD8), OX40L, and 4-1BBL. In some aspects, the RAPC expresses IL-21 (e.g., IL-21-Fc or IL-21-CD8), OX40L, IL-15 (e.g., IL-15-Fc or IL-15-CD8), and 4-1BBL.

In some aspects, the method comprises contacting the immune cells with (i) an RAPC expressing IL-21 (e.g., IL-21-Fc or IL-21-CD8) and OX40L and (ii) IL-15, wherein the RAPC does not express IL-15. In some aspects, the method comprises contacting the immune cells with (i) an RAPC expressing IL-21 (e.g., IL-21-Fc or IL-21-CD8) and OX40L and (ii) 4-1BBL, wherein the RAPC does not express 4-1BBL. In some aspects, the method comprises contacting the immune cells with (i) an RAPC expressing IL-21 (e.g., IL-21-Fc or IL-21-CD8) and OX40L, (ii) IL-15, and (iii) 4-1BBL; wherein the RAPC does not express IL-15 or 4-1BBL.

In some aspects, the method comprises contacting the immune cells with (i) an RAPC expressing IL-21 and IL-15 (e.g., IL-15-Fc or IL-15-CD8) and (ii) OX40L, wherein the RAPC does not express OX40L. In some aspects, the method comprises contacting the immune cells with (i) an RAPC expressing IL-21 and IL-15 (e.g., IL-15-Fc or IL-15-CD8) and (ii) 4-1BBL, wherein the RAPC does not express 4-1BBL. In some aspects, the method comprises contacting the immune cells with (i) an RAPC expressing IL-21 and IL-15 (e.g., IL-15-Fc or IL-15-CD8), (ii) OX40L, and (iii) 4-1BBL; wherein the RAPC does not express OX40L or 4-1BBL.

In some aspects, the method comprises contacting the immune cells with (i) an RAPC expressing IL-21 (e.g., IL-21-Fc or IL-21-CD8) and 4-1BBL and (ii) IL-15, wherein the RAPC does not express IL-15. In some aspects, the method comprises contacting the immune cells with (i) an RAPC expressing IL-21 (e.g., IL-21-Fc or IL-21-CD8) and 4-1BBL and (ii) OX40L, wherein the RAPC does not express OC-40L. In some aspects, the method comprises contacting the immune cells with (i) an RAPC expressing IL-21 (e.g., IL-21-Fc or IL-21-CD8) and 4-1BBL, (ii) IL-15, and (iii) OX40L; wherein the RAPC does not express IL-15 or OX40L.

Some aspects of the present disclosure are directed to a method of expanding a population of immune cells, e.g., T cells and/or NK cells, ex vivo, comprising contacting the population of immune cells with IL-21, e.g., soluble IL-21, IL-21-Fc, or IL-21-CD8, and OX40L. In some aspects, the method further comprises contacting the population of immune cells with an APC, e.g., an RAPC disclosed herein. In some aspects, the method further comprises contacting the population of immune cells, e.g., T cells and/or NK cells, with 4-1BBL. In some aspects, the method further comprises contacting the population of immune cells, e.g., T cells and/or NK cells, with IL-15, e.g., soluble IL-15, IL-15-Fc, or IL-15-CD8. In some aspects, the APC, e.g., RAPC, expresses IL-21 (e.g., IL-21-Fc or IL-21-CD8), OX40L, and 4-1BBL, and the population of immune cells, e.g., T cells and/or NK cells, is cultured in a medium comprising IL-15, e.g., soluble IL-15, IL-15-Fc, or IL-15-CD8. In some aspects, the APC, e.g., RAPC, expresses IL-21 (e.g., IL-21-Fc or IL-21-CD8), OX40L, and IL-15 (e.g., IL-15-Fc or IL-15-CD8), and the population of immune cells, e.g., T cells and/or NK cells, is cultured in a medium comprising 4-1BBL.

Some aspects of the present disclosure are directed to a method of expanding a population of immune cells, e.g., T cells and/or NK cells, ex vivo, comprising contacting the population of immune cells with IL-21 (e.g., soluble IL-21, IL-21-Fc, or IL-21-CD8) and a first APC, wherein the first APC expresses OX40L. In some aspects, the population of immune cells, e.g., T cells and/or NK cells, is cultured in a medium, wherein the medium comprises the IL-21 (e.g., soluble IL-21). In some aspects, the IL-21 is associated with a bead. In some aspects, the IL-21 (e.g., soluble IL-21, IL-21-Fc, and/or IL-21-CD8) is expressed by a second APC. In some aspects, the first RAPC and the second APC are different. In some aspects, the first APC and the second APC are the same.

In some aspects, the method further comprises contacting the population of immune cells, e.g., T cells and/or NK cells, with OX40L. In some aspects, the population of immune cells, e.g., T cells and/or NK cells, is cultured in a medium, wherein the medium comprises the OX40L. In some aspects, the OX40L is associated with a bead. In some aspects, the OX40L is expressed by a third APC. In some aspects, the third APC is different from the first APC and the second APC. In some aspects, the third APC is the same as the first APC, wherein the third APC is the same as the second APC, or wherein the third APC is the same as the first APC and the second APC.

In some aspects, the method further comprises contacting the population of immune cells, e.g., T cells and/or NK cells, with IL-15 (e.g., soluble IL-15, IL-15-Fc, or IL-15-CD8). In some aspects, the population of immune cells, e.g., T cells and/or NK cells, is cultured in a medium, wherein the medium comprises the IL-15 (e.g., soluble IL-15). In some aspects, the IL-15 is associated with a bead. In some aspects, the IL-15 is expressed by a fourth APC. In some aspects, the fourth APC is different from the first APC, the second APC, and the third APC. In some aspects, the fourth APC is the same as the first APC; wherein the fourth APC is the same as the second APC; wherein the fourth APC is the same as the third APC; wherein the fourth APC is the same as the first APC and the second APC; wherein the fourth APC is the same as the second APC and the third APC; wherein the fourth APC is the same as the first APC and the third APC; or wherein the fourth APC is the same as the first APC, the second APC, and the third APC.

In some aspects, the population of immune cells is expanded following contacting the immune cells with the RAPCs. In some aspects, contacting the population of immune cells with an RAPCs expressing (i) IL-21 (e.g., IL-21-Fc or IL-21-CD8) and 4-1BBL and (ii) IL-15 (e.g., IL-15-Fc or IL-15-CD8) and/or OX40L results in an increased expansion of the population of immune cells relative to the expansion of a population of immune cells not contacted with the RAPC or contacted with an APC not expressing (i) IL-21 (e.g., IL-21-Fc or IL-21-CD8) and 4-1BBL and (ii) IL-15 (e.g., IL-15-Fc or IL-15-CD8) and/or OX40L. In some aspects, the population of immune cells is expanded until the total number of immune cells in the population of immune cells is at least about 1×10⁷ to at least about 1×10¹², at least about 1×10⁸ to at least about 1×10¹², at least about 1×10⁹ to at least about 1×10¹², at least about 1×10⁸ to at least about 1×10¹¹, at least about 1×10⁹ to at least about 1×10¹¹, at least about 1×10¹⁰ to at least about 1×10¹¹, at least about 1×10⁷ to at least about 1×10¹⁰, at least about 1×10⁸ to at least about 1×10¹⁰, or at least about 1×10⁹ to at least about 1×10¹⁰ immune cells. In some aspects, the population of immune cells is expanded until the total number of immune cells in the population of immune cells is at least about 1×10⁷, at least about 5×10⁷, at least about 1×10⁸, at least about 5×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, at least about 5×10¹¹, at least about 6×10¹¹, at least about 7×10¹¹, at least about 8×10¹¹, at least about 9×10¹¹, or at least about 1×10¹² immune cells.

In some aspects, the method further comprises purifying the expanded population of immune cells. In some aspects, the purified population of immune cells following expansion are cryopreserved. Any method of cryopreserving immune cells can be used in the methods disclosed herein. In some aspects, the immune cells are cryopreserved in a solution (e.g., a medium) that comprises DMSO (e.g., 5-10% DMSO). In some aspects, the immune cells are thawed and administered to a subject in need thereof.

### III.A. Immune Cells

The methods disclosed herein can be used in the culture (e.g., expansion) of any population of immune cells. As such, some aspects of the present disclosure are directed to a method of expanding a population of immune cells ex vivo comprising contacting the population of immune cells with an RAPC disclosed herein. Any immune cell can be cultured according to the methods disclosed herein. In some aspects, the population of immune cells comprises one or more immune cell selected from a αβ T cell, a B cell, a natural killer (NK) cell, a tumor infiltrating lymphocyte (TIL), a pan γδ T cell, a Vδ1 γδ T cell, a Vδ2 γδ T cell, a Vδ3 γδ T cell, and any combination thereof. In some aspects, the T cell is selected from the group consisting of a naïve T cell, a stem cell memory T cell (Tscm), a central memory T cell (Tcm), an effector T cell, an effector memory T cell (Tem), a cytotoxic T cell, a helper T cell, and any combination thereof.

In some aspects, the population of immune cells comprises one or more immune cell obtained from a donor subject. In some aspects, the population of immune cells comprises one or more immune cell obtained from a donor human subject. In some aspects, the donor subject is afflicted with a disease or condition. In some aspects, the donor subject is afflicted with a cancer. In some aspects, the donor subject is afflicted with a cancer selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and any combination thereof. In certain aspects, the cancer comprises a lymphoma. In certain aspects, the cancer comprises a leukemia.

In some aspects, the immune cells are isolated from cord blood obtained from the subject. In some aspects, the immune cells are isolated from peripheral blood obtained from the subject. In some aspects, the population of immune cells comprises one or more gamma/delta T cells obtained from cord blood from a subject. In some aspects, the population of immune cells comprises one or more NK cells obtained from cord blood from a subject. In some aspects, the population of immune cells comprises one or more gamma/delta T cells and one or more NK cells obtained from cord blood from a subject. In some aspects, the population of immune cells comprises one or more gamma/delta T cells obtained from peripheral blood from a subject. In some aspects, the population of immune cells comprises one or more NK cells obtained from peripheral blood from a subject. In some aspects, the population of immune cells comprises one or more gamma/delta T cells and one or more NK cells obtained from peripheral blood from a subject.

In some aspects, the immune cells are isolated from a tumor sample obtained from a subject. Tumor biopsies often comprise a population of immune cells, including tumor infiltrating lymphocytes (TILs) and/or tumor associated macrophages (TAMs). Any methods can be used to obtain TILs and/or TAMs from a tumor sample, including, for example, the methods disclosed in U.S. Pat. No. 10,166,257, which is incorporated by reference herein in its entirety.

In some aspects, the donor subject is a healthy donor. In certain aspects, the donor subject is not afflicted with a cancer.

In some aspects, the population of immune cells comprises one or more T cells. In some aspects, the population of immune cells comprises one or more alpha/beta T cells, gamma/delta T cells, NK cells, or a combination thereof.

In some aspects, the population of immune cells comprises one or more immune cells derived from a stem cell. In some aspects, the population of immune cells comprises one or more immune cells derived from an induced pluripotent stem cell (iPSC). In some aspects, the population of immune cells comprises one or more immune cells derived from a hematopoietic stem cell. In some aspects, the population of immune cells comprises one or more immune cells derived from an embryonic stem cell.

In some aspects, the methods disclosed herein expand the number of immune cells in the population of immune cells. In some aspects, the methods disclosed herein expand the number of immune cells in the population of immune cells by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold by at least about day 8 of co-culture. In some aspects, the methods disclosed herein expand the number of immune cells in the population of immune cells by at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 225-fold, at least about 250-fold, at least about 275-fold, at least about 300-fold, or at least about 325-fold by at least about day 15 of co-culture. In some aspects, the methods disclosed herein expand the number of immune cells in the population of immune cells by at least about 200-fold, at least about 225-fold, at least about 250-fold, at least about 275-fold, at least about 300-fold, at least about 325-fold, at least about 350-fold, at least about 375-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold at least about 1000-fold, at least about 1050-fold, at least about 1100-fold, or at least about 1150-fold by at least about day 23 of co-culture. In some aspects, the methods disclosed herein expand the number of immune cells in the population of immune cells by at least about 900-fold, at least about 1000-fold at least about 1500-fold, at least about 2000-fold, at least about 2500-fold, at least about 3000-fold, at least about 3500-fold, at least about 4000-fold, at least about 4500-fold, at least about 5000-fold, at least about 5500-fold, at least about 6000-fold, at least about 6500-fold, or at least about 7000-fold by at least about day 27 of co-culture.

In some aspects, the methods disclosed herein expand the number of gamma/delta T cells in a mixed population of immune cells obtained from a subject. In some aspects, the percent of gamma/delta T cells in the expanded population of immune cells is at least about 30%, at least about 35%, at least about 40%, at least about 45%, at least about 50%, at least about 55%, at least about 60%, at least about 65%, at least about 70%, at least about 75%, at least about 85%, at least about 85%, at least about 90%, or at least about 95% of the total population of expanded population of immune cells. In some aspects, the methods disclosed herein expand the number of NK cells in a mixed population of immune cells obtained from a subject. In some aspects, the percent of NK cells in the expanded population of immune cells is at least about 30%, at least about 35%, at least about 40%, at least about 45%, at least about 50%, at least about 55%, at least about 60%, at least about 65%, at least about 70%, at least about 75%, at least about 85%, at least about 85%, at least about 90%, or at least about 95% of the total population of expanded population of immune cells.

In some aspects, cells expanded according to the methods disclosed herein result in a population of expanded cells comprising an increased percentage of immune cells having an NK cell surface phenotype. In some aspects, the NK cell surface phenotype comprises expression of one or more of CD56, CD16, CD30, CD94, and NKG2D. In some aspects, the NK cell surface phenotype comprises CD3− and CD56+ expression by the expanded NK cells. In some aspects, a higher percentage of expanded cells have an NK cell surface phenotype (e.g., CD3−CD56+) following co-incubation with one or more RAPCs expressing (i) IL-21 (e.g., IL-21-Fc or IL-21-CD8) and 4-1BBL and (ii) IL-15 (e.g., IL-15-Fc or IL-15-CD8) and/or OX40L as compared to the percentage of expanded cells having an NK cell surface phenotype following co-incubation with an APC not expressing (i) IL-21 (e.g., IL-21-Fc or IL-21-CD8) and 4-1BBL and (ii) IL-15 (e.g., IL-15-Fc or IL-15-CD8) and/or OX40L. In some aspects, the percent of total expanded cells expressing an NK cell surface phenotype (e.g., CD3−CD56+) following the co-incubation is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some aspects, the percent of total expanded cells expressing an NK cell surface phenotype (e.g., CD3-CD56+) following the co-incubation is at least about 50%. In some aspects, the percent of total expanded cells expressing an NK cell surface phenotype (e.g., CD3−CD56+) following the co-incubation is at least about 60%. In some aspects, the percent of total expanded cells expressing an NK cell surface phenotype (e.g., CD3−CD56+) following the co-incubation is at least about 70%. In some aspects, the percent of total expanded cells expressing an NK cell surface phenotype (e.g., CD3−CD56+) following the co-incubation is at least about 75%. In some aspects, the percent of total expanded cells expressing an NK cell surface phenotype (e.g., CD3−CD56+) following the co-incubation is at least about 80%. In some aspects, the percent of total expanded cells expressing an NK cell surface phenotype (e.g., CD3−CD56+) following the co-incubation is at least about 85%.

In some aspects, the percent of total expanded cells expressing a gamma/delta T cell surface phenotype (e.g., CD3+CD56−) following the co-incubation is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some aspects, the percent of total expanded cells expressing a gamma/delta T cell surface phenotype (e.g., CD3+CD56−) following the co-incubation is at least about 50%. In some aspects, the percent of total expanded cells expressing a gamma/delta T surface phenotype (e.g., CD3+CD56−) following the co-incubation is at least about 60%. In some aspects, the percent of total expanded cells expressing a gamma/delta T phenotype (e.g., CD3+CD56−) following the co-incubation is at least about 70%. In some aspects, the percent of total expanded cells expressing a gamma/delta T surface phenotype (e.g., CD3-CD56+) following the co-incubation is at least about 75%. In some aspects, the percent of total expanded cells expressing a gamma/delta T surface phenotype (e.g., CD3+CD56−) following the co-incubation is at least about 80%. In some aspects, the percent of total expanded cells expressing a gamma/delta T surface phenotype (e.g., CD3+CD56−) following the co-incubation is at least about 85%.

In some aspects, the immune cells are genetically modified. In some aspects, the immune cells are genetically modified (e.g., to introduce a CAR) before expansion using RAPCs described herein (e.g., in case of primary immune cells). In other aspects, the immune cells are genetically modified (e.g., to introduce a CAR) after expansion using RAPCs described herein (e.g., in case of iPSC derived immune cells). As such, some aspects of the present disclosure are directed to a method of expanding a population of genetically modified immune cells, comprising genetically modifying immune cells and contacting the genetically modified immune cells with an RAPC disclosed herein, thereby to expand the cells. Other aspects of the present disclosure are directed to a method of expanding a population of immune cells, comprising (i) contacting a population of immune cells with an RAPC disclosed herein to produce a population of expanded of immune cells, and (ii) genetically modifying one or more cell of the population of expanded immune cells.

Any means of genetic modification can be used in the compositions and methods disclosed herein. In some aspects, the immune cell, e.g., the expanded immune cell (e.g., an expanded gamma/delta T cell and/or an expanded NK cell), is genetically modified to comprise a chimeric antigen receptor. Chimeric antigen receptors, or CARs, are heterologous, recombinant receptors that can confer on a host cell, e.g., an immune cell, the ability to target a specific antigen. CARs can include, without limitation, artificial T-cell receptors, T-bodies, single-chain immunoreceptors, chimeric T-cell receptors, chimeric immunoreceptors, or any combination thereof. In some aspects, a CAR comprises an intracellular activation domain, a transmembrane domain, and an extracellular domain, wherein the extracellular domain comprises an antigen-binding domain.

In some aspects, the antigen-binding domain of the CAR comprises an antibody or an antigen-binding portion of an antibody. In some aspects, the antigen-binding domain of the CAR comprises an scFv, Fab, Fab′, Fv, F(ab′)2, dAb, or any fragments or combinations thereof. In some aspects, the antigen-binding domain of the CAR specifically binds an antigen. In some aspects, the antigen-binding domain of the CAR specifically binds one or more antigen on a tumor cell. In some aspects, the antigen-binding domain of the CAR specifically binds one or more antigen selected from CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), and any combination thereof.

In some aspects, the CAR comprises a transmembrane domain. Any transmembrane domain can be used in the methods and compositions disclosed herein. In some aspects, the CAR comprises a transmembrane domain derived from a transmembrane molecule that is a co-receptor on immune cells or a transmembrane domain of a member of the immunoglobulin superfamily. In some aspects, the CAR comprises a transmembrane domain derived from a transmembrane domain of CD28, CD8 alpha, CD4, or CD19.

In some aspects, the CAR comprises one or more costimulatory signaling regions. In some aspects, the costimulatory signaling region comprises a signaling region of CD28, OX-40, 41BB, CD27, inducible T cell costimulator (ICOS), CD3 gamma, CD3 delta, CD3 epsilon, CD247, Ig alpha (CD79a), Fc gamma receptor, FcR, CD137, DAP10, or any combination thereof. In some aspects, the CAR further comprises a CD3 zeta signaling domain.

In some aspects, the CAR further comprises a hinge region. In some aspects, the CAR comprises a hinge region derived from the hinge region of IgG1, IgG2, IgG3, IgG4, IgA, IgD, IgE, IgM, CD28, CD8 alpha, or any combination thereof. In some aspects, the CAR comprises an IgG4 hinge region.

In some aspects, the immune cell, e.g., the expanded immune cell (e.g., an expanded gamma/delta T cell and/or an expanded NK cell), is genetically modified to express a heterologous T cell receptor (TCR). In some aspects, the heterologous TCR is capable of binding one or more antigen present on a tumor cell. In some aspects, the heterologous TCR is capable of binding an antigen selected from the group consisting of CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), and any combination thereof.

In some aspects, the expanded immune cells are harvested (e.g., isolated and/or purified) from the cell culture. The expanded immune cells can be harvested using any methods. In some aspects, the expanded immune cells are harvested by positive or negative selection including, but not limited to, the use of antibodies. In some aspects, expanded T cells are harvested using antibodies directed to CD2, CD3, CD4, CD5, CD8, CD14, CD16, CD19 and/or CD25. In some aspects, the expanded immune cells are harvested using cell sorting, e.g., flow cytometry. In some aspects, the expanded immune cells are harvested with the irradiated RAPCs.

### III.B. Methods of Treatment

Certain aspects of the present disclosure are directed to a method of treating a disease or condition in a subject in need thereof, comprising expanding a population of immune cells ex vivo by contacting the population of immune cells with an RAPC or a population of RAPCs disclosed herein; and administering the expanded population of immune cells to the subject. In some aspects, the method further comprises isolating the expanded population of cells prior to administering the cells to the subject. In some aspects, the expanded population of cells is cryopreserved, as disclosed herein, prior to administering to the subject.

In some aspects, the disease or condition comprises a cancer. In some aspects, the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. In some aspects, the cancer comprises a lymphoma. In some aspects, the cancer comprises a leukemia.

In some aspects, the method further comprises pretreating the subject prior to administering the population of immune cells. In some aspects, the subject is administered a chemotherapy prior to administering the population of immune cells. In some aspects, the subject is administered an immuno-depleting chemotherapy prior to administering the population of immune cells. In some aspects, the immuno-depleting chemotherapy comprises cyclophosphamide, fludarabine, or both.

In some aspects, the method comprises administering to the subject (i) the population of expanded cells and (ii) a cytokine. In some aspects, the cytokine comprises IL-2, an analog thereof, a variant thereof, or a fragment thereof.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1. Recombinant Antigen Presenting Cell Generation

K562 cells (ATCC) were cultured in RPMI (Gibco) containing 10% FBS (Hyclone). Human 4-1BB ligand (4-1BBL), OX40 ligand (OX40L), membrane bound IL-15 (mIL-15), and membrane bound IL-21 (mIL-21) sequences were synthesized de novo and cloned into lentiviral plasmids with the EF1A promoter for packaging into LentiOne lentiviral vectors (GegTech) (FIGS. 1A-1D). RAPC1 was generated by transducing 4-1BBL into K562 cells; RAPC2 was generated by transducing 4-1BBL and mIL-15 into K562 cells; RAPC3 was generated by transducing 4-1BBL and mIL-21 into K562 cells; and RAPC4 was generated by transducing 4-1BBL, mIL-15, mIL-21, and OX40L into K562 cells. The cells were subsequently purified using MACS magnetic bead-based cell sorting according to manufacturer's recommendations by labeling with APC conjugated antibodies and anti-APC microbeads (FIG. 1E). All four lines were selected for 4-1BBL-expressing cells with an anti-4-1BBL antibody. After recovery culture, RAPC2 and RAPC4 were subsequently selected for IL-15-expressing cells using an anti-IL-15 antibody, and RAPC3 and RAPC4 were selected for IL-21-expressing cells using an anti-IL-21 antibody. Finally, RAPC4 were selected for OX40L-expressing cells using an anti-OX40L antibody. RAPC cells were allowed to recover in culture after each sequential round of selection. Initially, RAPCs were treated with 15 μg/mL Mitomycin C for four hours and washed twice prior to co-incubation with peripheral blood (PB)-derived and cord blood (CB)-derived gamma delta T (gdT) and natural killer (NK) cells. Alternatively, for some experiments, RAPCs were irradiated before co-incubation with NK cells by X-Ray irradiation on the XRAD-iR160 (Precision) with 100 Gy, dosed with four cycles of 25 Gy per cycle.

### Isolation and Expansion of Peripheral Blood and Cord Blood Derived gdT and NK Cells

Gama delta (gd) T cells were positively selected and NK cells were negatively selected via immunomagnetic bead separation from frozen peripheral blood (PB) or cord blood (CB) (STEMCELL Technologies). Selected gdT and NK cells were coincubated with mitomycin C-pretreated RAPC2 or RAPC4 feeder cells in RPMI (Gibco) containing 10% FBS (Corning). gdT cultures were supplemented with IL-2 (Peprotech, 100 IU/mL) and IL-21 (Peprotech, 30 ng/ml), and NK cultures were supplemented with IL-2 (Peprotech, 100 IU/mL). Cell cultures were refreshed with half-volume medium changes supplemented with IL-2 and/or IL-21 every two days. Cells were cultured for four weeks in G-Rex 6M well plate (Wilson Wolf) and feeder cell activation by co-incubation was repeated every 7 days.

### Expansion of NK Cells in Additional Experiments

NK cells were activated with irradiated RAPC1 or RAPC4 feeders at a 2:1 ratio (RAPC:NK) by co-incubation in IMDM (Gibco) containing 15% FBS (Gibco). Culture medium for RAPC1 feeders was supplemented with IL-2 (Peprotech, 10 ng/ml), IL-7 (Peprotech, 10 ng/ml), and IL-15 (Peprotech, 10 ng/ml); culture medium for RAPC4 co-incubation was supplemented with IL-2 (10 ng/ml) and IL-7 (10 ng/ml). Cells were cultured for three days and then expanded for four additional days in a G-Rex 24 well plate with a 60% medium change on the fifth day of culture. This activation and expansion cycle was repeated three times for up to four total rounds of activation.

### Flow Cytometry

CB-derived and PB-derived NK cells activated with Mitomycin C treated RAPCs were stained with anti-CD3 and anti-CD56 and run on the FACS Canto (BD). NK cells in additional experiments were stained with anti-CD3, anti-CD56, anti-CD16, anti-CD30, anti-CD94, anti-NKG2D, or anti-NKp46 and were analyzed by flow cytometry to determine cell surface expression of all markers.

### In Vitro Cytotoxicity Assay

CB-derived and PB-derived gdT and NK cells activated with RAPCs were coincubated with Capan2 (pancreatic adenocarcinoma cells), GSU (gastric cancer cells), HCT116 (colorectal carcinoma cells), HT55 (colorectal carcinoma cells), K562 (chronic myelogenous leukemia cells), Mia Paca2 (pancreatic carcinoma cells), Nalm6 (acute lymphoblastic leukemia cells), OvCar3 (ovarian adenocarcinoma cells), Raji (Burkitt's lymphoma cells), or HT29VEC (colorectal carcinoma cells) (ATCC) at effector to target ratios of 10:1, 3:1, 1:1, or 0.3:1 for 24 hours. Percent killing was determined for each cell line by a flow cytometry-based assay. For additional NK cell experiments, NK cells were harvested on day 6 of the second round of activation by RAPC4 and coincubated with Capan2, GSU, HCT116, Raji, or HUVEC cells (ATCC) at effector to target ratios of 3:1, 1:1, 0.3:1, or 0.1:1 for 24 hours. Percent killing was determined by Cell Titer Glo Assay (Promega).

### Example 2. Immune Cells Co-incubated with RAPCs Have Improved Properties

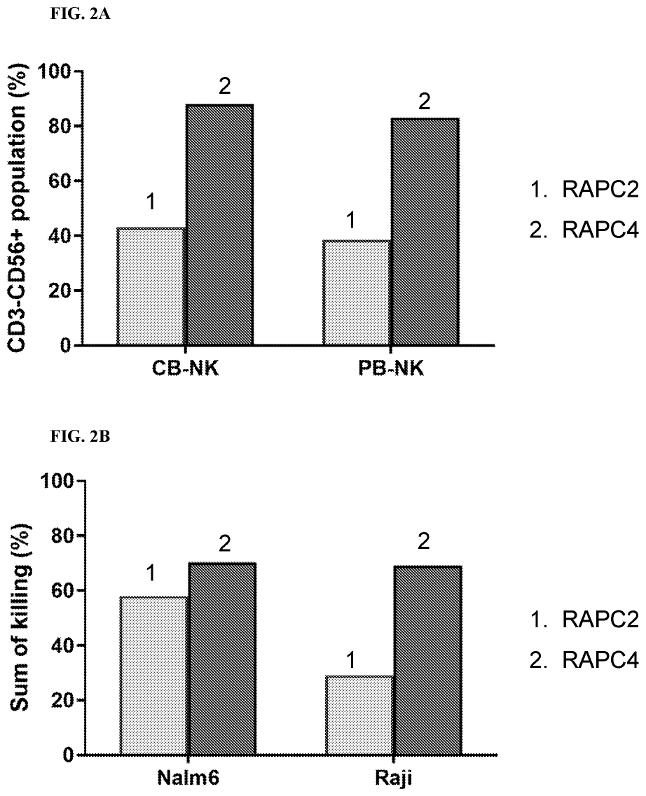

RAPC lines RAPC1, RAPC2, RAPC3, and RAPC4 were successfully generated using the approach described above to transduce K562 cells with lentiviral vector and sequentially purify for 41BBL, OX40L, mIL-15, and/or mIL-21 (FIGS. 1A-1E). Co-incubation of PB-derived or CB-derived NK cells with Mitomycin C treated RAPC2 or RAPC4 cells resulted in >100-fold expansion of cells from four sequential rounds of activation and expansion (Table 2); CB-derived NK cells co-incubated with RAPC4 had the highest cumulative expansion at 6830-fold expansion of all cells tested. In addition to higher overall cell expansion, both CB-derived and PB-derived NK cells co-incubated with RAPC4 resulted in higher percentages of cells with an NK cell surface phenotype (CD3-CD56+) compared to cells co-incubated with RAPC2 cells (FIG. 2A) and had higher average cell killing efficacy against Nalm6 and Raji after 24 hours (FIG. 2B).

TABLE 2
Total cell growth (fold-expansion) for CB-NK and
PB-NK co-incubated with RAPC2 and RAPC4.

| | CB-NK + | PB-NK + | CB-NK + | PB-NK + |
|---|---|---|---|---|
| | RAPC2 | RAPC2 | RAPC4 | RAPC4 |
| Day 0 | 1 | 1 | 1 | 1 |
| Day 8 | 3.17 | 2.39 | 12.32 | 7.73 |
| Day 15 | 18.76 | 6.14 | 308.29 | 53.94 |
| Day 23 | 299.67 | 31.05 | 1187.89 | 209.26 |
| Day 27 | 1892.6 | 178.19 | 6830.91 | 939.11 |

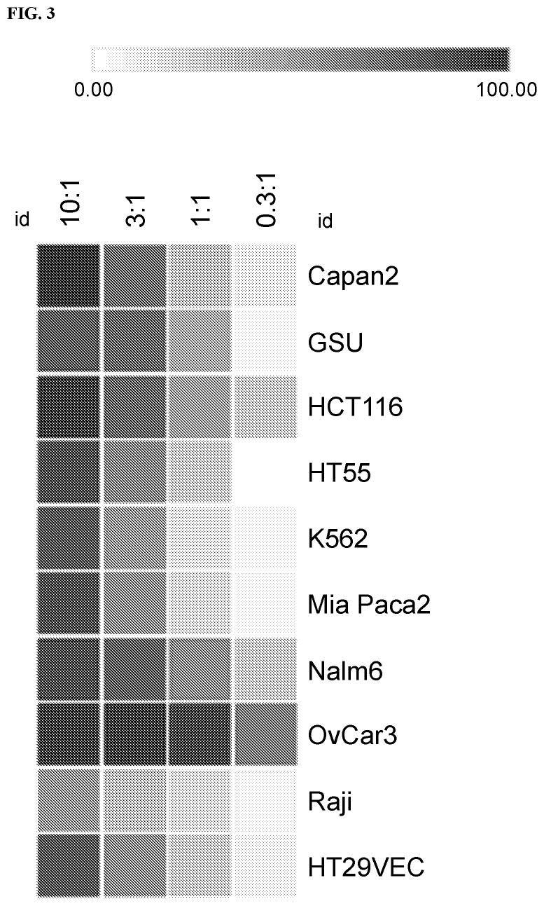

Co-incubation of PB-derived or CB-derived gdT cells with Mitomycin C treated RAPC2 or RAPC4 cells resulted in >1,000-fold expansion of cells from four sequential rounds of activation and expansion with continued expansion through four rounds of activation and expansion (Table 3). CB-derived gdT cells activated with Mitomycin C treated RAPC4 demonstrated intrinsic cytolytic activity against multiple cell lines (FIG. 3).

TABLE 3
Cell expansion of cord blood (CB) and peripheral blood (PB)-derived
gdT cells by co-incubation with RAPC2 and RAPC4 feeder cells.

| | CB-gdT + | PB-gdT + | CB-gdT + | PB-gdT + |
|---|---|---|---|---|
| | RAPC2 | RAPC2 | RAPC4 | RAPC4 |
| Day 0 | 1 | 1 | 1 | 1 |
| Day 8 | 127.14 | 108.23 | 64.8 | 76.45 |
| Day 15 | 612.21 | 1120.18 | 545.98 | 496.12 |
| Day 23 | 1963.93 | 4529.9 | 2009.49 | 2164.51 |
| Day 27 | 10461.04 | 24903.85 | 9970.5 | 15775.99 |

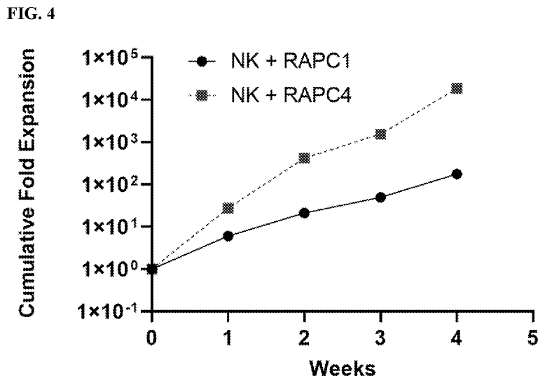

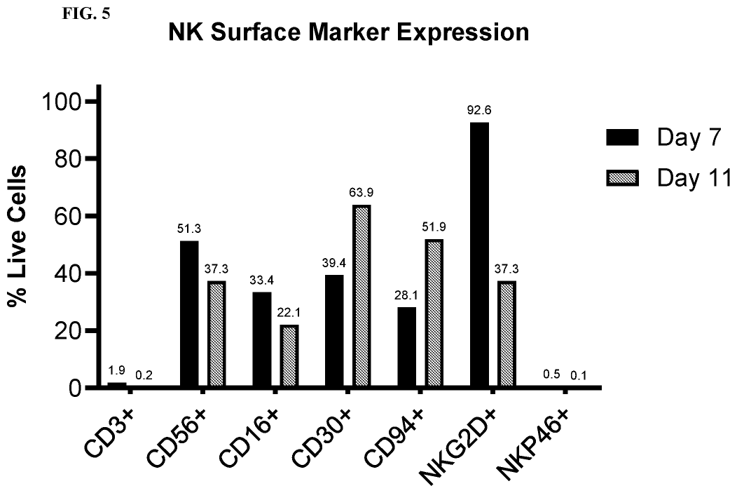

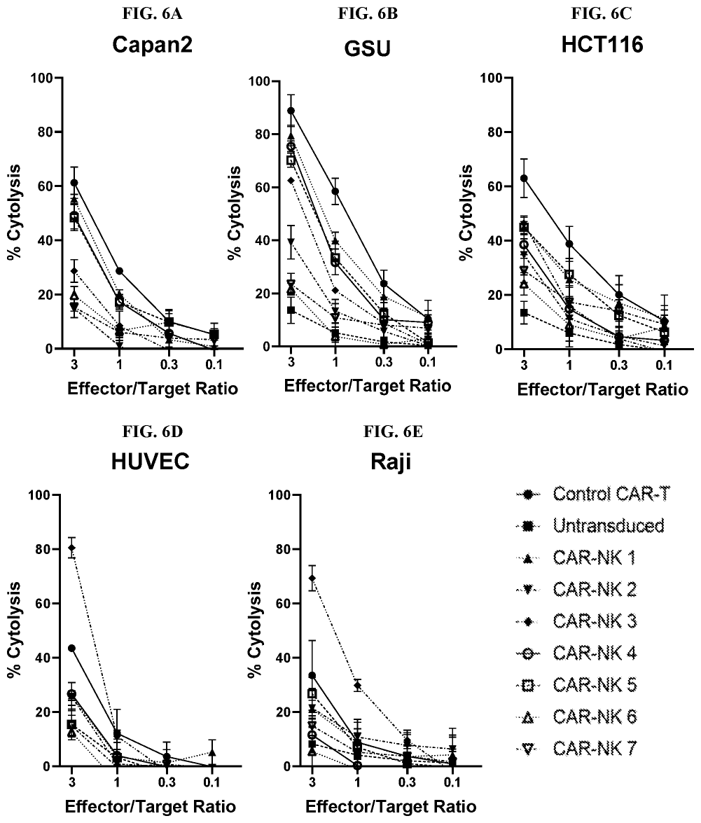

In additional experiments, NK cells were coincubated with RAPC1 and RAPC4 cells that were irradiated with 100 Gy total exposure, dosed with four cycles of 25 Gy irradiation. Under these culture conditions, NK cells coincubated with 2:1 RAPC4:NK cell ratio expanded >15,000-fold over four sequential rounds of activation and expansion, higher than expansion achieved by NK cells coincubated with RAPC1 feeder cells (FIG. 4). Cell surface markers were assessed in NK cells activated by co-incubation with irradiated RAPC4 cells, and the population had low expression of CD3 and NKp46, with high expression of NK cell markers CD56, CD16, CD30, CD94, and NKG2D when assessed at day 7 and day 11 post-activation (FIG. 5). NK cells activated with RAPC4 feeders were subsequently transduced with seven different CAR lentiviral vectors and cell expansion was similar between transduced and untransduced (UTD) cells (Table 4). Additionally, NK cells coincbuated with irradiated RAPC4 demonstrated CAR-mediated killing of multiple cancer cell lines including Capan2 (FIG. 6A), GSU (FIG. 6B), and HCT116 (FIG. 6C) cells depending on the CAR included.

TABLE 4
Total cell growth (fold-expansion) for CAR-NK
cells coincubated with irradiated RAPC4.

| | | CAR- | CAR- | CAR- | CAR- | CAR- | CAR- | CAR- |
|---|---|---|---|---|---|---|---|---|
| Day | UTD | NK 1 | NK 2 | NK 3 | NK 4 | NK 5 | NK 6 | NK 7 |
| 4 | 1.2 | 1.2 | 1.2 | 1.2 | 1.2 | 1.2 | 1.2 | 1.2 |
| 7 | 2.2 | 2 | 2.4 | 2.2 | 2.4 | 2.2 | 2 | 1.9 |
| 9 | 6.9 | 6.7 | 7.9 | 7.5 | 6.7 | 7.1 | 6.6 | 6.9 |
| 11 | 21.8 | 17.3 | 22 | 21.5 | 23.5 | 24.3 | 18.1 | 21 |
| 14 | 92 | 81.3 | 84.7 | 77.3 | 82 | 99.3 | 99.3 | 90.3 |
| 18 | 1352.4 | 1219.5 | 1101.1 | 950.79 | 1033.2 | 1251.18 | 1181.67 | 1065.54 |
| 21 | 5648.8 | 5699.13 | 4336.64 | 4406.1 | 2837.2 | 4508.22 | 4101.09 | 4027.38 |

RAPC feeder cells activated and supported robust expansion of both gdT and NK cells isolated from multiple sources. The RAPCs were effective when used after Mitomycin C treatment or X-Ray irradiation was used to block cell proliferation and under multiple culture conditions. Both gdT and NK cells demonstrated expected cell surface phenotypes and were effective in cell killing assays.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed is:

1. A recombinant antigen presenting cell (RAPC), which comprises (i) one or more nucleic acid molecules encoding IL-21 and (ii) one or more nucleic acid molecules encoding OX40L.

2. The RAPC of claim 1, which further comprises (iii) one or more nucleic acid molecule encoding 4-1BBL.

3. The RAPC of claim 1 or 2, which further comprises (iv) one or more nucleic acid molecules encoding IL-15.

4. The RAPC of any one of claims 1 to 3, which expresses (i) IL-21 and (ii) OX40L.

5. The RAPC of any one of claims 1 to 4, which expresses (iii) 4-1BBL.

6. The RAPC of any one of claims 1 to 5, which expresses (iv) IL-15.

7. The RAPC of any one of claims 1 to 6, wherein the IL-21 is a membrane bound IL-21.

8. The RAPC of claim 7, wherein the membrane bound IL-21 is a fusion protein comprising a human IL-21 polypeptide and an Fc region of an immunoglobulin.

9. The RAPC of claim 7 or 8, wherein the membrane bound IL-21 comprises (i) the human IL-21 polypeptide, (ii) a hinge region, (iii) the Fc region of an immunoglobulin, and (iv) a transmembrane domain.

10. The RAPC of any one of claims 6 to 9, wherein the IL-15 is a membrane bound IL-15.

11. The RAPC of claim 10, wherein the membrane bound IL-15 is a fusion protein comprising a human IL-15 polypeptide and an Fc region of an immunoglobulin.

12. The RAPC of claim 10 or 11, wherein the membrane bound IL-15 comprises (i) the human IL-15 polypeptide, (ii) a hinge region, (iii) the Fc region of an immunoglobulin, and (iv) a transmembrane domain.

13. The RAPC of any one of claims 8 to 12, wherein the Fc region is an IgG1, IgG2, IgG3, or IgG4 Fc region.

14. The RAPC of any one of claims 8 to 13, wherein the Fc region is an IgG4 Fc region.

15. The RAPC of any one of claims 12 to 14, wherein the hinge region comprises an immunoglobulin hinge region or a modified immunoglobulin hinge region.

16. The RAPC of any one of claims 12 to 15, wherein the hinge region comprises an IgG1, IgG2, IgG3, IgG4 or CD8 hinge region.

17. The RAPC of any one of claims 12 to 16, wherein the transmembrane domain comprises a CD4 or CD8 transmembrane domain.

18. The RAPC of any one of claims 12 to 17, which comprises a dendritic cell, an engineered clonal cell line, a naturally derived cancer cell line, a bead-based artificial antigen presenting cell, an artificial lipid vesicle or other lipid bilayer containing system, an exosome, or a solid support having one or more of IL-21, IL-15, OX-40L and 4-1BBL attached thereto.

19. The RAPC of claim 18, wherein the solid support is selected from a bead, a spherical or non-spherical nanoparticle support, a carbon nanotube support, a magnetic particle support, a filamentous polymer support, a two dimensional support or any combination thereof.

20. The RAPC of any one of claims 1 to 19, which is a K562 cell.

21. The RAPC of any one of claims 1 to 20, which is a genetically modified K562 cell.

22. A population of RAPCs comprising the RAPC of any one of claims 1 to 21.

23. A population of RAPCs comprising a first RAPC and a second RAPC, wherein:
(a) the first RAPC and the second RAPC comprise one or more nucleic acid molecules encoding IL-21 and one or more nucleic acid molecules encoding OX40L; or
(b) the first RAPC comprises one or more nucleic acid molecules encoding IL-21, and wherein the second RAPC expresses one or more nucleic acid molecules encoding OX40L.

24. The population of RAPCs of claim 23, wherein the first RAPC, the second RAPC, or both further comprises one or more nucleic acid molecules encoding 4-1BBL.

25. The population of RAPCs of claim 23 or 21, wherein the first RAPC, the second RAPC, or both further comprises one or more nucleic acid molecules encoding IL-15.

26. The population RAPCs of any one of claims 23 to 25, further comprising a third RAPC.

27. The population RAPCs of claim 26, wherein the third RAPC comprises one or more nucleic acid molecules encoding 4-1BBL.

28. The population of RAPCs of claim 26 or 27, wherein the third RAPC comprises one or more nucleic acid molecules encoding IL-15.

29. The population RAPCs of any one of claims 23 to 28, further comprising a fourth RAPC.

30. The population RAPCs of claim 29, wherein the fourth RAPC comprises one or more nucleic acid molecules encoding IL-15.

31. The population of RAPCs of claim 29 or 30, wherein the first RAPC, the second RAPC, the third RAPC, the fourth RAPC, or any combination thereof comprises a dendritic cell, an engineered clonal cell line, a naturally derived cancer cell line, a bead-based artificial antigen presenting cell, an artificial lipid vesicle or other lipid bilayer containing system, an exosome, a solid support, or any combination thereof.

32. The population of RAPCs of claim 31, wherein the solid support comprises a bead, a spherical or non-spherical nanoparticle support, a carbon nanotube support, a magnetic particle support, a filamentous polymer support, a two dimensional support, or any combination thereof.

33. The population of RAPCs of any one of claims 29 to 32, wherein the first RAPC, the second RAPC, the third RAPC, the fourth RAPC, or any combination thereof is a K562 cell.

34. The population of RAPCs of any one of claims 29 to 33, wherein the first RAPC, the second RAPC, the third RAPC, the fourth RAPC, or any combination thereof is a genetically modified K562 cell.

35. A method of expanding a population of immune cells ex vivo, comprising contacting the population of immune cells with the RAPC of any one of claims 1 to 21 or the population of RAPCs of any one of claims 22 to 34.

36. A method of expanding a population of immune cells ex vivo, comprising contacting the population of immune cells with an RAPC, wherein the RAPC expresses IL-21, OX40L, 4-1BBL, and IL-15.

37. The method of claim 35 or 36, wherein the immune cells comprise an αβ T cell, a B cell, a natural killer (NK) cell, a tumor infiltrating lymphocyte (TIL), a pan γδ T cell, a Vδ1 γδ T cell, a Vδ2 γδ T cell, a Vδ3 γδ T cell or any combination thereof.

38. The method of claim 37, wherein the immune cell is selected from the group consisting of a naïve T cell, a stem cell memory T cell (Tscm), a central memory T cell (Tcm), an effector T cell, an effector memory T cell (Tem), a cytotoxic T cell, a helper T cell, and any combination thereof.

39. The method of any one of claims 35 to 38, wherein the population of immune cells comprises one or more genetically modified immune cells.

40. The method of any one of claims 35 to 39, wherein the population of immune cells is genetically modified before the population of immune cells is contacted with the RAPC.

41. The method of any one of claims 35 to 40, wherein the population of immune cells are genetically modified after the population of immune cells is contacted with the RAPC.

42. The method of any one of claims 35 to 41, wherein the population of immune cells comprises one or more immune cells derived from an induced pluripotent stem cell (iPSC).

43. The method of any one of claims 39 to 42, wherein the genetically modified immune cells comprise a chimeric antigen receptor (CAR), an engineered T cell receptor (TCR), or a combination thereof.

44. The method of claim 43, wherein the CAR or the TCR is capable of binding one or more antigens present on a tumor cell.

45. The method of claim 43 or 44, wherein the CAR or the TCR is capable of binding an antigen selected from the group consisting of CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII, VEGFR2, HER2-HER3 in combination, HER1-HER2 in combination, NY-ESO-1, synovial sarcoma X breakpoint 2 (SSX2), melanoma antigen (MAGE), melanoma antigen recognized by T cells 1 (MART-1), gp100, prostate specific antigen (PSA), prostate specific membrane antigen (PSMA), prostate stem cell antigen (PSCA), and any combination thereof.

46. The method of any one of claims 35 to 45, wherein the population of immune cells is expanded until the total number of immune cells in the population of immune cells is at least about 1×10⁷ to at least about 1×10¹², at least about 1×10⁸ to at least about 1×10¹², at least about 1×10⁹ to at least about 1×10¹², at least about 1×10⁸ to at least about 1×10¹¹, at least about 1×10⁹ to at least about 1×10¹¹, at least about 1×10¹⁰ to at least about 1×10¹¹, at least about 1×10⁷ to at least about 1×10¹⁰, at least about 1×10⁸ to at least about 1×10¹⁰, or at least about 1×10⁹ to at least about 1×10¹⁰ immune cells.

47. The method of any one of claims 35 to 46, wherein the population of immune cells is expanded until the total number of immune cells in the population of immune cells is at least about 1×10⁷, at least about 5×10⁷, at least about 1×10⁸, at least about 5×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, at least about 5×10¹¹, at least about 6×10¹¹, at least about 7×10¹¹, at least about 8×10¹¹, at least about 9×10¹¹, or at least about 1×10¹² immune cells.

48. The method of any one of claims 35 to 47, wherein the population of immune cells are isolated from a donor subject.

49. The method of claim 48, wherein the donor subject is a human.

50. The method of claim 48 or 49, wherein the donor subject is afflicted with a cancer.

51. The method of any one of claims 35 to 50, further comprising purifying an expanded population of immune cells.

52. The method of 51, wherein the expanded population of immune cells comprises a population of alpha/beta T cells, gamma/delta T cells, NK cells or a combination thereof.

53. The method of claim 51 or 52, further comprising administering the expanded population of immune cells to a subject in need thereof.

54. A method of treating a disease or condition in a subject in need thereof, comprising (i) expanding a population of immune cells ex vivo by contacting the population of immune cells with the RAPC of any one of claims 1 to 21 or the population of RAPCs of any one of claims 22 to 34; (ii) purifying an expanded population of immune cells; and (iii) administering the expanded population of immune cells to the subject.

55. The method of claim 53 or 54, wherein the subject is afflicted with a cancer.

56. The method of claim 50 or 55, wherein the cancer comprises bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof.

57. The method of any one of claims 50, 55, and 56, wherein the cancer comprises a lymphoma or a leukemia.

58. A method of expanding a population of immune cells ex vivo, comprising contacting the population of immune cells with interleukin-21 (IL-21) and OX40L.

59. The method of claim 58, further comprising contacting the population of immune cells with a recombinant antigen-presenting cell (RAPC).

60. A method of expanding a population of immune cells ex vivo, comprising contacting the population of T cells with an RAPC, IL-21, and OX40L.

61. The method of any one of claims 58 to 60, further comprising contacting the population of immune cells with 4-1BB ligand (4-1BBL).

62. The method of any one of claims 58 to 61, further comprising contacting the population of immune cells with interleukin-15 (IL-15).

63. The method of any one of claims 59 to 62, wherein the RAPC expresses IL-21, OX40L, 4-1BBL, IL-15, or any combination thereof.

64. The method of any one of claims 59 to 63, wherein the RAPC expresses IL-21.

65. The method of any one of claims 59 to 64, wherein the RAPC expresses OX40L.

66. The method of any one of claims 59 to 65, wherein the RAPC expresses 4-1BBL.

67. The method of any one of claims 59 to 66, wherein the RAPC expresses IL-15.

68. The method of any one of claims 59 to 67, wherein the RAPC expresses IL-21 and OX40L, wherein the population of immune cells are cultured in a medium, and wherein the medium comprises 4-1BBL and IL-15.

69. The method of any one of claims 59 to 68, wherein the RAPC expresses IL-21, OX40L, and 4-1BBL, wherein the population of immune cells are cultured in a medium, and wherein the medium comprises IL-15.

70. The method of any one of claims 59 to 68, wherein the RAPC expresses IL-21, OX40L, and IL-15, wherein the population of immune cells are cultured in a medium, and wherein the medium comprises 4-1BBL.

71. The method of any one of claims 59 to 70, wherein the RAPC comprises a dendritic cell, an engineered clonal cell line, a naturally derived cancer cell line, a bead-based artificial antigen presenting cell, an artificial lipid vesicle or other lipid bilayer containing system, an exosome, a solid support, or any combination thereof.

72. The method of claim 71, wherein the solid support comprises a bead, a spherical or non-spherical nanoparticle support, a carbon nanotube support, a magnetic particle support, a filamentous polymer support, a two dimensional support or any combination thereof.

73. The method of any one of claims 59 to 71, wherein the RAPC is a genetically modified K562 cell.

74. A method of expanding a population of immune cells ex vivo, comprising contacting the population of T cells with IL-21 and a first RAPC, wherein the first RAPC expresses OX40L.

75. The method of claim 74, wherein the population of immune cells is cultured in a medium, wherein the medium comprises the IL-21.

76. The method of claim 75, wherein the IL-21 is associated with a bead.

77. The method of claim 74, wherein the IL-21 is expressed by a second RAPC.

78. The method of claim 77, wherein the first RAPC and the second RAPC are different.

79. The method of claim 77 or 78, wherein the first RAPC and the second RAPC are the same.

80. The method of any one of claims 74 to 79, further comprising contacting the population of immune cells with OX40L.

81. The method of claim 80, wherein the population of immune cells is cultured in a medium, wherein the medium comprises the OX40L.

82. The method of claim 81, wherein the OX40L is associated with a bead.

83. The method of claim 80, wherein the OX40L is expressed by a third RAPC.

84. The method of claim 83, wherein the third RAPC is different from the first RAPC and the second RAPC.

85. The method of claim 83, wherein the third RAPC is the same as the first RAPC, wherein the third RAPC is the same as the second RAPC, or wherein the third RAPC is the same as the first RAPC and the second RAPC.

86. The method of any one of claims 74 to 85, further comprising contacting the population of immune cells with IL-15.

87. The method of claim 86, wherein the population of immune cells is cultured in a medium, wherein the medium comprises the IL-15.

88. The method of claim 87, wherein the IL-15 is associated with a bead.

89. The method of claim 86, wherein the IL-15 is expressed by a fourth RAPC.

90. The method of claim 89, wherein the fourth APC is different from the first RAPC, the second RAPC, and the third RAPC.

91. The method of claim 89 wherein the fourth RAPC is the same as the first RAPC; wherein the fourth RAPC is the same as the second RAPC; wherein the fourth RAPC is the same as the third RAPC; wherein the fourth RAPC is the same as the first RAPC and the second RAPC; wherein the fourth RAPC is the same as the second RAPC and the third RAPC; wherein the fourth RAPC is the same as the first RAPC and the third RAPC; or wherein the fourth RAPC is the same as the first RAPC, the second RAPC, and the third RAPC.

92. A method of expanding a population of immune cells ex vivo, comprising contacting the population of T cells with an RAPC, wherein the RAPC expresses IL-21, OX40L, 4-1BBL, and IL-15.

93. The method of any one of claims 74 to 92, wherein one or more of the first RAPC, the second RAPC, the third RAPC, or the fourth RAPC comprises a dendritic cell, an engineered clonal cell line, a naturally derived cancer cell line, a bead-based artificial antigen presenting cell, an artificial lipid vesicle or other lipid bilayer containing system, an exosome, a solid support, or any combination thereof.

94. The method of claim 93, wherein the solid support comprises a bead, a spherical or non-spherical nanoparticle support, a carbon nanotube support, a magnetic particle support, a filamentous polymer support, a two dimensional support, or any combination thereof.

95. The method of any one of claims 74 to 94, wherein the RAPC is a genetically modified K562 cell.

96. The method of any one of claims 58 to 95, wherein the IL-21 is a fusion protein comprising a human IL-21 polypeptide and an Fc region of an immunoglobulin.

97. The method of claim 96, wherein the IL-21 comprises (i) the human IL-21 polypeptide, (ii) a hinge region, (iii) the Fc region of an immunoglobulin, and (iv) a transmembrane domain.

98. The method of any one of claims 63 to 73 and 86 to 97, wherein the IL-15 is a fusion protein comprising a human IL-15 polypeptide and an Fc region of an immunoglobulin.

99. The method of claim 98, wherein the IL-15 comprises (i) the human IL-15 polypeptide, (ii) a hinge region, (iii) the Fc region of an immunoglobulin, and (iv) a transmembrane domain.

100. The method of any one of claims 96 to 99, wherein the Fc region is an IgG1, IgG2, IgG3, or IgG4 Fc region.

101. The method of any one of claims 96 to 100, wherein the Fc region is an IgG4 Fc region.

102. The method of any one of claims 97 to 101, wherein the hinge region comprises an immunoglobulin hinge region or a modified immunoglobulin hinge region.

103. The method of any one of claims 97 to 102, wherein the hinge region comprises an IgG1, IgG2, IgG3, IgG4 or CD8 hinge region.

104. The method of any one of claims 97 to 103, wherein the transmembrane domain comprises a CD4 or CD8 transmembrane domain.

105. The method of any one of claims 58 to 104, wherein the population of immune cells is expanded until the total number of T cells in the population of T cells is at least about 1×10⁷ to at least about 1×10¹², at least about 1×10⁸ to at least about 1×10¹², at least about 1×10⁹ to at least about 1×10¹², at least about 1×10⁸ to at least about 1×10¹¹, at least about 1×10⁹ to at least about 1×10¹¹, at least about 1×10¹⁰ to at least about 1×10¹¹, at least about 1×10⁷ to at least about 1×10¹⁰, at least about 1×10⁸ to at least about 1×10¹⁰, or at least about 1×10⁹ to at least about 1×10¹⁰ T cells.

106. The method of any one of claims 58 to 104, wherein the population of immune cells is expanded until the total number of T cells in the population of T cells is at least about 1×10⁷, at least about 5×10⁷, at least about 1×10⁸, at least about 5×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, at least about 5×10¹¹, at least about 6×10¹¹, at least about 7×10¹¹, at least about 8×10¹¹, at least about 9×10¹¹, or at least about 1×10¹² T cells.

107. The method of any one of claims 58 to 106, wherein the population of immune cells are obtained from a donor subject.

108. The method of claim 107, wherein the donor subject is a human.

109. The method of claim 107 or 108, wherein the donor subject is afflicted with a cancer.

110. The method of any one of claims 58 to 109, further comprising administering the population of immune cells to a subject in need thereof.

111. The method of any one of claims 58 to 110, wherein the immune cells comprise an αβ T cell, a B cell, a natural killer (NK) cell, a tumor infiltrating lymphocyte (TIL), a pan γδ T cell, a Vδ1 γδ T cell, a Vδ2 γδ T cell, a Vδ3 γδ T cell or any combination thereof.
